(19) United States
(12) Patent Application Publication
(10) Pub. No.: US 2023/0137667 A1
(43) Pub. Date: May 4, 2023

(54) METHODS AND COMPOSITIONS FOR TREATMENT OF CORONAVIRUS INFECTION

(71) Applicants: The University of North Carolina at Chapel Hill, Chapel Hill, NC (US); Battelle Memorial Institute, Richland, WA (US)

(72) Inventors: Ralph Baric, Haw River, NC (US); Amy Sims, Richland, WA (US); Hugh D. Mitchell, Richland, WA (US); Katrina M. Waters, Richland, WA (US)

(21) Appl. No.: 17/906,560

(22) PCT Filed: Mar. 19, 2021
(86) PCT No.: PCT/US2021/023189

§ 371 Date: Sep. 16, 2022

Related U.S. Application Data
(60) Provisional application No. 62/991,818, filed on Mar. 19, 2020.

(51) Int. Cl. A61K 31/7056; A61K 31/662; A61K 31/7076; A61P 31/14

(52) U.S. Cl. CPC A61K 31/7056; A61K 31/662; A61K 31/7076; A61P 31/14

(57) ABSTRACT

The present invention provides methods and compositions for treating coronavirus infection and diseases and disorders associated with or caused by coronavirus infection.

### STATEMENT OF PRIORITY

This application claims the benefit, under 35 U.S.C. § 119(e), of U.S. Provisional Application No. 62/991,818, filed on Mar. 19, 2020, the entire contents of which are incorporated by reference herein.

### STATEMENT OF GOVERNMENT SUPPORT

This invention was made with government support under Grant No. AI106772 awarded by the National Institutes of Health. The government has certain rights in the invention.

### FIELD OF THE INVENTION

The present invention relates generally to the fields of virology, infectious disease and medicine. More specifically, the invention relates to methods of treating coronavirus infections in humans.

### BACKGROUND OF THE INVENTION

Coronaviruses (CoV) are important emerging pathogens associated with severe disease outcomes in humans and animals causing significant global morbidity and mortality. In 2003, severe acute respiratory coronavirus (SARS-CoV) emerged from closely related bat CoV strains, spread to 26 nations, and caused approximately 8,000 human cases and 800 deaths, worldwide. In 2012, Middle East respiratory syndrome CoV (MERS-CoV) emerged, causing approximately 2,400 cases with a ˜35% mortality rate in 27 countries and the outbreak is still ongoing. SARS- and MERS-CoV disease severity are strongly influenced by aging and other co-morbidities (e.g., diabetes, obesity) and mortality rates exceed 50% in aged individuals (>60 years). These data underscore the highly pathogenic potential of emerging CoVs and reinforce the critical need for comprehensive experimental approaches that define pathogenic programs which could be targeted for antiviral therapy to reverse severe disease outcomes.

Acute Respiratory Distress Syndrome (ARDS) is a severe end stage lung disease characterized by rapid onset respiratory failure resulting from diffuse alveolar damage, vascular leakage into the airways and pulmonary fibrosis. ARDS is a complex syndrome with multiple etiologies which complicates diagnosis and treatment. While not the most prevalent cause, severe respiratory infections can progress to ARDS including those by highly pathogenic avian influenza (H5N1) and both SARS- and MERS-CoV. Dissecting the mechanisms of MERS-CoV-induced ARDS has been hampered by human genetic variation, variable co-morbidities and limited human sample availability. The present invention overcomes previous shortcomings in the art by providing methods and compositions for treating diseases and disorders associated with coronavirus infection.

### SUMMARY OF THE INVENTION

In one embodiment, the present invention provides a method for treating a coronavirus infection in a subject, comprising administering to the subject a therapeutically effective amount of an inhibitor of an unfolded protein response (UPR), an inhibitor of an integrated stress response (ISR), and/or an inhibitor of protein kinase RNA-like endoplasmic reticulum kinase (PERK).

In a further embodiment, the present invention provides a method for treating a disease or disorder caused by or associated with a coronavirus infection in a subject, comprising administering to the subject a therapeutically effective amount of an inhibitor of an unfolded protein response (UPR), an inhibitor of an integrated stress response (ISR), and/or an inhibitor of protein kinase RNA-like endoplasmic reticulum kinase (PERK).

Other and further aspects, features, benefits, and advantages of the present invention will be apparent from the following description of the embodiments of the invention given for the purpose of disclosure.

### BRIEF DESCRIPTION OF THE DRAWINGS

FIG. 1 panels A-E show transcriptomics and proteomics data plots. In FIG. 1 panels A-C, donor matched microvascular endothelial cells (MVE) human airway epithelial cell cultures (HAE) and fibroblasts (FB) were infected with wild type MERS-CoV (MOI of 5) and supernatants collected at the indicated times and viral titers determined by plaque assay. Results are shown as plaque forming units (PFU) per mL over time. Each data point represents averaged data from supernatant collected from 10 different wells (5 wells harvested for RNA and 5 wells fractionated for proteins, and lipids). The graphs in FIG. 1 panels A, B and C show levels of replication detected for all three tissue donors in all three cell types. Error bars indicate standard deviation from the mean. FIG. 1 panel D shows a heat map of functional enrichment performed on transcriptomic data from all three donor samples in both cell types. Results were only retained that were present in all three donors, keeping only the least significant score. In this way a true consensus response is represented by all indicated functions. FIG. 1 panel E shows a heat map of functional enrichment performed on proteomic data from all three donor samples in both cell types. Results were only retained that were present in all three donors, as in FIG. 1 panel D. The blue arrow highlights proteins in the hemoglobin complex and red asterisks highlight apoptotic proteins from the endoplasmic reticulum.

FIG. 2 panels A-D show heat maps of lipidomics and cell viability assessment. FIG. 2 panel A shows a heat map of enrichment analysis using lipid classes as enrichment sets. TG-triglycerides, Cer-ceramide, CL-cardiolipins, CE-cholesterol esters, PA-phosphatidic acid, GalCer-galactosylceramide, GM3-GM3 ganglioside, PI-phosphatidylinositol, DG-diglycerides, PS-phosphatidylserine, PG-phosphatidylglycerol, SM-sphingomyelin, PE-phosphatidylethanolamine, PC-phosphatidylcholine. FIG. 2 panel B shows a heat map of abundance of individual lipid species, whose fold change compared to mock-infected samples was at a p-value of 0.001 or below in at least one condition and was a member of one of the indicated lipid classes (triglycerides or ceramides). FIG. 2 panels C and D show bar graphs of cell viability following MERS-CoV infection. Donor matched MVE and FB were infected with wild type MERS-CoV and assessed for cell viability using the CellTiter-Glo kit according to manufacturer's instructions (Promega) at the indicated times post infection. Relative light units were graphed over time and error bars indicate standard deviation from the mean. Statistical analysis performed in GraphPad determined by Mann-Whitney U test. Gray bars—mock infected cells, red bars—MERS-CoV infected MVE, blue bars—MERS-CoV infected FB, ****p=<0.0001, **p=<0.002, ns not statistically significant.

FIG. 3 panels A-D show bar graphs of 3 Caspases 3/7 activation following MERS-CoV infection of primary human lung MVE but not primary lung FB. Donor matched MVE and FB were plated and mock-infected or infected with UV-inactivated or wild type MERS-CoV (MOI 5) and at 24 and 48 hours post infection caspase 3/7 activation or cell viability was determined according to manufacturer's instructions using the Apotox Triplex kit (each well was measured for both parameters). Uninfected cells were treated with staurosporin (8 MVE/10 μM FB) to determine the maximal amount of caspase 3/7 activation. Uninfected cells were treated with ionomycin (50 μM MVE/60 μM FB) to demonstrate loss of cell viability via non-apoptotic pathway. Results are graphed as relative light units and error bars indicate standard deviation from the mean. Statistical analysis performed in GraphPad determined by Mann-Whitney U test. FIG. 3 panels A and B show bar graphs of results from caspase 3/7 activation. FIG. 3 panels C and D show bar graphs of results from cell viability assay. Solid red bars—MVE 24 hour samples, open red bars—MVE 48 hour samples, solid blue bars—FB 24 hour samples, open blue bars—FB 48 hour samples. ****p=<0.0001, ***p=<0.0002, ns—not statistically significant.

FIG. 4 panels A-F show bar graphs of protein markers which suggest activation of unfolded protein response (UPR) in MVE. FIG. 4 panels A-C show bar graphs of protein expression of markers of the unfolded protein response. Each figure shows the expression behavior of a UPR marker in FB (left three groups) and MVE (right three groups), with individual donors represented separately as individual shaded hues. For each donor, a time course of expression is shown that includes samples taken at 0, 12, 24, 36 and 48 hours post infection. Values are expressed as the log 10 p-value of the change between the infection and control conditions, with sign assigned according to the direction of fold change. FIG. 4 panels D-F show bar graphs of viral protein expression. MERS-CoV spike, open reading frame 4a (ORF4a) and membrane protein abundances are represented as the ratio of each protein to the average abundance of all other proteins detected in each respective sample.

FIG. 5 panels A-D show data plots of increased caspase 3/7 activation following treatment with trans-ISRIB and MERS-CoV infection of MVE and FB. In FIG. 5 panels A and B, donor matched uninfected FB (circles, FIG. 5 panel A) and MVE (circles, FIG. 5 panel B) were treated with serial dilutions of trans-ISRIB inhibitor (2.5 μM to 0.00488 μM) and cell viability assessed at 48 hours post treatment using Promega's CellTiter Glo kit according to manufacturer's instructions. Each circle represents mean values from two experiments and are graphed as percent toxicity. Error bars indicate standard deviation from the mean. In parallel, the same donor matched FB (squares, FIG. 5 panel A) and MVE (squares, FIG. 5 panel B) were infected with MERS-nanoluc and simultaneously treated with the same serial dilutions of trans-ISRIB. Nanoluciferase expression was assayed at 48 hours post infection using Promega's NanoGlo kit according to manufacturer's instructions. Control wells were either treated with drug diluent and UV-inactivated virus or infected with MERS-nanoluc (MOI 5) and treated with only drug diluent. Each square represents mean values from two experiments and are graphed as percent inhibition. Error bars indicate standard deviation from the mean. FIG. 5 panel A squares—MERS-nanoluc infected FB treated with trans-ISRIB serial dilutions, FIG. 5 panel A circles—uninfected FB treated with trans-ISRIB serial dilutions, FIG. 5 panel B squares—MERS-nanoluc infected MVE treated with trans-ISRIB serial dilutions, FIG. 5 panel B circles—uninfected MVE treated with trans-ISRIB serial dilutions, trans-integrated stress response inhibitor (ISRIB), MVE primary human lung microvascular endothelial cell, FB primary human lung fibroblast, MERS-nanoluc MERS-CoV expressing nanoluciferase In FIG. 5 panels C and D, donor matched FB (FIG. 5 panel C) and MVE (FIG. 5 panel D) were plated and infected with UV-inactivated or wild type MERS-CoV and at 48 hours post infection caspase 3/7 activation was determined according to manufacturer's instructions using the Apotox Triplex kit. Control uninfected cells were treated with staurosporin (8 μM MVE/10 μM FB, activates caspase 3/7) or ionomycin (50 μM MVE/60 μM FB, does not activate caspase 3/7). Results are graphed as relative light units and error bars indicate standard deviation from the mean. Statistical analysis performed in GraphPad determined by Mann-Whitney U test. FIG. 5 panel C bars-MERS-CoV infected FB treated with indicated compound, FIG. 5 panel D bars-MERS-CoV infected MVE treated with indicated compound

FIG. 6 panels A-D show data plots of PERK inhibitor reducing MERS-CoV replication in infected primary human lung MVE and FB. Donor matched uninfected FB (circles, FIG. 6 panels A and B) and MVE (circles, FIG. 6 panels C and D) were treated with serial dilutions of PERK inhibitor (100 μM to 2 μM) and cell viability assessed at 24 (FIG. 6 panels A and C) and 48 (FIG. 6 panels B and D) hours post treatment using Promega's CellTiter Glo kit according to manufacturer's instructions. Each circle represents mean values from two experiments and are graphed as percent toxicity. Error bars indicate standard deviation from the mean. In parallel, the same donor matched FB (squares, FIG. 6 panels A and B) and MVE (squares, FIG. 6 panels C and D) were infected with MERS-nanoluc and simultaneously treated with the same serial dilutions of PERK inhibitor. Nanoluciferase expression was assayed at 24 (squares, FIG. 6 panels A and C) and 48 (squares, FIG. 6 panels B and D) hours post infection using Promega's NanoGlo kit according to manufacturer's instructions. Control wells were either treated with drug diluent and UV-inactivated virus or infected with MERS-nanoluc (MOI 5) and treated with only drug diluent. Each square represents mean values from two experiments and are graphed as percent inhibition. Error bars indicate standard deviation from the mean. FIG. 6 panels A and B squares—MERS-nanoluc infected FB treated with PERK inhibitor serial dilutions, FIG. 6 panels A and B circles—uninfected FB treated with PERK inhibitor serial dilutions, FIG. 6 panels C and D squares—MERS-nanoluc infected MVE treated with PERK inhibitor serial dilutions, FIG. 6 panels C and D circles—uninfected MVE treated with PERK inhibitor serial dilutions. PERK protein kinase R-like ER kinase, MVE primary human lung microvascular endothelial cell, FB primary human lung fibroblast, MERS-nanoluc MERS-CoV expressing nanoluciferase.

FIG. 7 panels A-F show data plots of PERK inhibitor AMG PERK 44 treatment decreasing MERS-CoV pathogenesis. hDPP4 mice were treated with PERK AMG PERK 44 or sham control and infected with 5×10⁴ PFU of MERS-CoV MAm35c4 or PBS control. Weight loss was observed over the course of a seven day infection (FIG. 7 panel A) and viral titers (FIG. 7 panel B) and pulmonary hemorrhage was scored at the time of harvest (FIG. 7 panel C). Daily respiratory function measurements (FIG. 7 panels D-F) were taken via whole body plethysmography. Error bars indicate standard error of the mean, # indicates a p value of <0.1 and * indicates a p value of <0.05.

FIG. 8 panels A-E show data plots and histology of AMG PERK 44 reducing clinical signs of acute lung injury in MERS-CoV challenge model. The histological features of acute lung injury were blindly scored using the American Thoracic Society Lung Injury Scoring system by Matute-Bello, creating an aggregate score for: neutrophils in the alveolar and interstitial space, hyaline membranes, proteinaceous debris filling the air spaces, and alveolar septal thickening. FIG. 8 panels A and B show data plots of scoring for ALI and diffuse alveolar damage in male and female mice, respectively. Representative lung pathology for (FIG. 8 panel C) mock-infected, (FIG. 8 panel D) MERS-CoV infected and sham treated and (FIG. 8 panel E) MERS-CoV infected and AMG PERK 44 treated male mice at 7 days post infection. * indicates a p value of <0.001.

### DETAILED DESCRIPTION OF THE INVENTION

The present invention will now be described with reference to the following embodiments and examples. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference herein in their entirety.

The present invention is based on the unexpected discovery of inhibitors that can be used to treat a coronavirus infection in a subject, as well as a disease or disorder caused by, or associated with infection by a coronavirus. Thus, in one embodiment, the present invention provides a method for treating a coronavirus infection in a subject, comprising administering to the subject a therapeutically effective amount of an inhibitor of an unfolded protein response (UPR), an inhibitor of an integrated stress response (ISR), and/or an inhibitor of protein kinase RNA-like endoplasmic reticulum kinase (PERK).

The present invention also provides a method for treating a disease or disorder caused by or associated with a coronavirus infection in a subject, comprising administering to the subject a therapeutically effective amount of an inhibitor of an unfolded protein response (UPR), an inhibitor of an integrated stress response (ISR), and/or an inhibitor of protein kinase RNA-like endoplasmic reticulum kinase (PERK).

In some embodiments, the coronavirus can be Middle East respiratory syndrome coronavirus (MERS-CoV), severe acute respiratory syndrome coronavirus (SARS-CoV) or severe acute respiratory syndrome coronavirus 2 (SARS-CoV-2). At least seven strains of human coronaviruses have been identified, including human coronavirus 229E (HCoV-229E), human coronavirus 0C43 (HCoV-0C43), severe acute respiratory syndrome coronavirus (SARS-CoV), human coronavirus NL63 (HCoV-NL63, New Haven coronavirus), human coronavirus HKU1, Middle East respiratory syndrome-related coronavirus (MERS-CoV), previously known as novel coronavirus 2012 and HCoV-EMC, and severe acute respiratory syndrome coronavirus 2 (SARS-CoV-2), previously known as 2019-nCoV or “novel coronavirus 2019.” The methods of this invention can be used to treat infections and diseases caused by any of these human coronaviruses.

Studies have shown that human coronaviruses can vary significantly in risk factor. Some can kill more than 30% of those infected (such as MERS-CoV), and some are relatively harmless, such as the common cold. Coronaviruses cause colds with major symptoms, such as fever, and sore throat from swollen adenoids, occurring primarily in the winter and early spring seasons. Coronaviruses can cause pneumonia (either direct viral pneumonia or a secondary bacterial pneumonia) and bronchitis (either direct viral bronchitis or a secondary bacterial bronchitis). SARS-CoV, which causes severe acute respiratory syndrome (SARS), has a unique pathogenesis because it causes both upper and lower respiratory tract infections.

Coronaviruses primarily infect the upper respiratory and gastrointestinal tract of mammals and birds. They also cause a range of diseases in farm animals and domesticated pets, some of which can be serious and are a threat to the farming industry. In chickens, the infectious bronchitis virus (IBV), a coronavirus, targets not only the respiratory tract but also the urogenital tract. The virus can spread to different organs throughout the chicken. Economically significant coronaviruses of farm animals include porcine coronavirus (transmissible gastroenteritis coronavirus, TGE) and bovine coronavirus, which both result in diarrhea in young animals. Feline coronavirus: two forms, feline enteric coronavirus is a pathogen of minor clinical significance, but spontaneous mutation of this virus can result in feline infectious peritonitis (FIP), a disease associated with high mortality. Similarly, there are two types of coronavirus that infect ferrets: Ferret enteric coronavirus causes a gastrointestinal syndrome known as epizootic catarrhal enteritis (ECE), and a more lethal systemic version of the virus (like FIP in cats) known as ferret systemic coronavirus (FSC). There are two types of canine coronavirus (CCoV), one that causes mild gastrointestinal disease and one that has been found to cause respiratory disease. Mouse hepatitis virus (MHV) is a coronavirus that causes an epidemic murine illness with high mortality, especially among colonies of laboratory mice. Some strains of MHV cause a progressive demyelinating encephalitis in mice which has been used as a murine model for multiple sclerosis. Sialodacryoadenitis virus (SDAV) is highly infectious coronavirus of laboratory rats, which can be transmitted between individuals by direct contact and indirectly by aerosol. Acute infections have high morbidity and tropism for the salivary, lachrymal and harderian glands. A HKU2-related bat coronavirus called swine acute diarrhea syndrome coronavirus (SADS-CoV) causes diarrhea in pigs. The methods of this invention can be used to treat infections and diseases in animals caused by any of these coronaviruses.

In some embodiments, the inhibitor employed in the methods of this invention can be GSK2606414 (PERK inhibitor), GSK2656157 (PERK inhibitor), ISRIB (trans-isomer) (PERK inhibitor), Salubrinal inhibitor of eIF-2a dephosphorylation and inhibitor of stress-mediated apoptosis), Sal003 (inhibitor of eIL-2a phosphatase), Azoramide (inhibitor of unfolded protein response (UPR)), AMG PERK 44 (PERK inhibitor), PERK-IN-2 (PERK inhibitor), PERK-IN-3 (PERK inhibitor), 4μ8C-CAS 14003-96-4-Calbiochem (IRE1 inhibitor III), CAS 608512-97-6-Calbiochem (PKR inhibitor), STF-083010-Calbiochem (IRE1 inhibitor), KIRA6-Calbiochem (IRE1 inhibitor), LDN-0070977 (PERK inhibitor III), NMS-E194, AMG-44, trazodone HCl, any derivative of any of said inhibitors, and any combination of said inhibitors and derivatives.

In some embodiments, an inhibitor of this invention can be an inhibitor of activating transcription factor 6 (ATF6), such as PP1-13, PP1-14 or PP1-19 (Liu et al. “High content screening identifies inhibition of nuclear translocation of ATF6” Intl. J. Mol. Med. 37(2):407-414 (2016)).

In some embodiments, an inhibitor of this invention can be an antibody or antibody fragment that binds a target of this invention, e.g., PERK, eIL-2α phosphatase, ATF6, IRE1.

In some embodiments of this invention, the inhibitor can inhibit viral activity by at least 10%, 20%, 30%, 40%, 50% 60%, 70%, 80%, 90% or 100%.

In some embodiments, the inhibitor is administered in a dosage range of about 0.1 μg/kg per day to about 500 mg/kg per day, including any value within this range even if not explicitly set forth herein. Nonlimiting examples of a dosage range include a value at the lower end of the range of about 0.1 μg/kg, 0.5 μg/kg, 1.0 μg/kg, 10 μg/kg, 20 μg/kg, 30 μg/kg, 40 μg/kg, 50 μg/kg, 100 μg/kg, 200 μg/kg, 300 μg/kg, 400 μg/kg, 500 μg/kg, 600 μg/kg, 700 μg/kg, 900 μg/kg, 1.0 mg/kg, 2.0 mg/kg, 3.0 mg/kg, 4.0 mg/kg, 5.0 mg/kg, 10 mg/kg, 20 mg/kg, 30 mg/kg, 40 mg/kg, 50 mg/kg, 60 mg/kg, 70 mg/kg, 80 mg/kg, 90 mg/kg, or 100 mg/kg, and a value at the upper end of the range of about 500 mg/kg, 450 mg/kg, 400 mg/kg, 350 mg/kg, 300 mg/kg or 200 mg/kg.

As is well known in the art, the methods of the present invention may be administered alone or in combination with one or more other antiviral agents to a subject to treat a particular condition. Thus, in some embodiments, the methods of this invention further comprise administering an antiviral drug.

Nonlimiting examples of an antiviral drug of this invention include interferon, ribavirin, adefovir, tenofovir, acyclovir, brivudin, cidofovir, fomivirsen, foscarnet, ganciclovir, penciclovir, amantadine, rimantadine and/or zanamivir, as well as any other antiviral drug now know or later identified, any of which can be administered singly or in any combination.

In treatments described herein, effective doses of the agents of this invention can be administered in compositions, i.e., they may be administered together (i.e., simultaneously), but may also be administered separately or sequentially. In general, combination therapy is typically administered together, the rationale being that such simultaneous administration induces multiple simultaneous stresses on the virus. The specific dosages given will depend on absorption, inactivation and excretion rate of the agents as well as other factors. It is to be noted that dosage values will also vary with the severity of the condition to be alleviated.

The terms “co-administration” or “combined administration” or “administered in combination with” or the like as utilized herein are meant to encompass administration of the selected therapeutic agents to a single patient, and are intended to include treatment regimens in which the agents are not necessarily administered by the same route of administration or at the same time. Fixed combinations are also within the scope of the present disclosure. The administration of a pharmaceutical combination of this invention results in a beneficial effect, e.g., a synergistic or additive therapeutic effect, compared to a monotherapy applying only one of its pharmaceutically active ingredients or as compared to the current standard of care therapy. The treatment used in the methods described herein may be administered by any conventional route, as described herein. One or more components may be administered parentally, e.g., in the form of injectable solutions or suspensions, or in the form of injectable deposit formulations.

It is contemplated that in some embodiments of this invention, the inhibitor and/or active agent can be administered in the form of a nucleic acid molecule. Thus, in some embodiments, the treatment methods of this invention can comprise administering to the subject a nucleic acid molecule comprising a nucleotide sequence encoding the inhibitor and/or other active agent.

The efficacy of the treatments described herein may be monitored using standard protocols. For example, treatment may be followed by a determination of the amount of coronavirus in a sample of the subject that may contain virus, e.g., by detection of coronavirus RNA in a biological sample (e.g., serum, plasma, urine, feces, CSF, bodily fluid or exudate, sputum, saliva, etc.) from the subject or patient.

### Definitions

The singular forms “a,” “an” and “the” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence or between the two ends (e.g., between domains) such that the function of the polynucleotide or polypeptide is not materially altered. The total of ten or less additional nucleotides or amino acids includes the total number of additional nucleotides or amino acids added together. The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in biological activity of at least about 50% or more as compared to the activity of a polypeptide consisting of the recited sequence.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc., as if each such subcombination is expressly set forth herein. Moreover, such language also indicates that one or more of the specified amino acids can be disclaimed. For example, in particular embodiments the amino acid is not A, G or I; is not A; is not G or V; etc., as if each such possible disclaimer is expressly set forth herein.

As used herein, the terms “reduce,” “reduces,” “reduction” and similar terms mean a decrease of at least about 5%, 10%, 15%, 20%, 25%, 35%, 50%, 75%, 80%, 85%, 90%, 95%, 97%, 98%, 99%, 100% or more.

As used herein, the terms “enhance,” “enhances,” “enhancement” and similar terms indicate an increase of at least about 10%, 20%, 25%, 50%, 75%, 100%, 150%, 200%, 300%, 400%, 500% or more.

As used herein, the term “polypeptide” encompasses both peptides and proteins, unless indicated otherwise.

A “polynucleotide,” “nucleic acid,” or “nucleic acid molecule” as used herein is a sequence of nucleotide bases, and may be RNA, DNA or DNA-RNA hybrid sequences (including both naturally occurring and non-naturally occurring nucleotide), but in representative embodiments are either single or double stranded DNA sequences.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” nucleotide is enriched by at least about 10-fold, 100-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

As used herein, by “isolate” or “purify” (or grammatical equivalents) a virus vector, it is meant that the virus vector is at least partially separated from at least some of the other components in the starting material. In representative embodiments an “isolated” or “purified” virus vector is enriched by at least about 10-fold, 100-fold, 1000-fold, 10,000-fold or more as compared with the starting material.

A “therapeutic polypeptide” is a polypeptide or peptide that can alleviate, reduce, prevent, delay and/or stabilize symptoms that result from an absence or defect in a protein in a cell or subject and/or is a polypeptide that otherwise confers a benefit to a subject.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

A “treatment effective,” “therapeutic,” or “effective” amount as used herein is an amount that is sufficient to provide some improvement or benefit to the subject. Alternatively stated, a “treatment effective,” “therapeutic,” or “effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion.

PERK (eukaryotic translation initiation factor 2-a kinase 3, eIF2α kinase 3, PEK, WRS) is a Ser/Thr protein kinase, type I membrane protein of the endoplasmic reticulum that is abundant in secretory tissues and is inducible by ER-stress. Protein kinase R (PKR)-like endoplasmic reticulum kinase (PERK) is a type I endoplasmic reticulum transmembrane protein containing a stress-sensing domain facing the endoplasmic reticulum lumen and a cytosolic kinase domain. PERK is a major component of the unfolded protein response (UPR), which promotes the adaptation of cells to various forms of stress. PERK is activated in response to a variety of endoplasmic reticulum stresses implicated in numerous disease states. PERK regulates proliferation of beta cells during embryonic and neonatal development and is essential for viability of acinar cells in mouse exocrine pancreas, neither of which is associated with endoplasmic reticulum stress response. PERK is also required for endoplasmic reticulum functions including proinsulin trafficking and quality control in beta cells. Similarly, PERK modulates proliferation and differentiation of osteoblasts as well as secretion of type I collagen. PERK phosphorylates the a subunit of the translation initiation factor eIF2 at serine 51, a modification that plays a key role in the regulation of mRNA translation in stressed cells. PERK-eIF2α phosphorylation pathway maintains insulin biosynthesis and glucose homeostasis, facilitates tumor formation and decreases the efficacy of tumor treatment with chemotherapeutic drugs.

Nonlimiting examples of an inhibitor of this invention include GSK2606414 (PERK inhibitor), GSK2656157 (PERK inhibitor), ISRIB (trans-isomer) (PERK inhibitor), Salubrinal inhibitor of eIF-2α dephosphorylation and inhibitor of stress-mediated apoptosis), Sal003 (inhibitor of eIL-2α phosphatase), Azoramide (inhibitor of unfolded protein response (UPR)), AMG PERK 44 (PERK inhibitor), PERK-IN-2 (PERK inhibitor), PERK-IN-3 (PERK inhibitor), 4μ8C-CAS 14003-96-4-Calbiochem (IRE1 inhibitor III), CAS 608512-97-6-Calbiochem (PKR inhibitor), STF-083010-Calbiochem (IRE1 inhibitor), KIRA6-Calbiochem (IRE1 inhibitor), LDN-0070977 (PERK inhibitor III), NMS-E194, and AMG-44, including any derivatives thereof, as described for example in the following patent publications: US20190241573, WO2019021208, WO2017046738, WO2017046737, US2019037570, US20190194135, US20190135772 and WO2017220477, the entire contents of each of which are incorporated by reference herein.

As used herein a “coronavirus infection” or “infection by a coronavirus” means an infection, including a patient being infected, with any coronavirus virus now known or later identified, including, but not limited to, human coronavirus HCoV-NL63, HCoV-0C43, HCoV-229E, HCoV-HKU1, SARS-CoV (severe acute respiratory syndrome coronavirus), the causative agent of severe acute respiratory syndrome (SARS), MERS-CoV (Middle East respiratory syndrome coronavirus, or EMC/2012), the causative agent of Middle East respiratory syndrome (MERS), and SARS-CoV-2 (severe acute respiratory syndrome coronavirus 2), the causative agent of coronavirus disease 2019 (COVID-19).

### Subjects, Pharmaceutical Formulations, and Modes of Administration

The inhibitors, derivatives and other active agents according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, lagomorphs, etc. Human subjects include in utero (e.g., embryos, fetuses), neonates, infants, juveniles, adults and geriatric subjects. The terms “subject” and “patient” can be used interchangeably in some embodiments of this invention and do not denote a particular age or sex. The subject or patient can be any animal susceptible to infection by a coronavirus.

In representative embodiments, the subject is “in need of” the methods of the invention and thus in some embodiments can be a “subject in need thereof.”

In particular embodiments, the present invention provides a pharmaceutical composition comprising an inhibitor, derivative and/or active agent of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

By “pharmaceutically acceptable” it is meant a material that is not toxic or otherwise undesirable, i.e., the material may be administered to a subject without causing any undesirable biological effects.

In particular embodiments, more than one administration (e.g., two, three, four or more administrations) may be employed to achieve the desired therapeutic effect over a period of various intervals, e.g., hourly, daily, weekly, monthly, yearly, etc.

Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). The most suitable route in any given case will depend on the nature and severity of the condition being treated and/or prevented and on the nature of the particular agent that is being used.

Injectables can be prepared in conventional forms, either as liquid solutions or suspensions, solid forms suitable for solution or suspension in liquid prior to injection, or as emulsions. Alternatively, one may administer the agents of the invention in a local rather than systemic manner, for example, in a depot or sustained-release formulation. Further, the agents can be delivered adhered to a surgically implantable matrix (e.g., as described in U.S. Patent Publication No. 20040013645).

The inhibitors, derivatives and active agents disclosed herein can be administered to the lungs of a subject by any suitable means, optionally by administering them as particles an aerosol suspension, which the subject inhales. The respirable particles can be liquid or solid. Aerosols of liquid particles comprising the agents of this invention may be produced by any suitable means, such as with a pressure-driven aerosol nebulizer or an ultrasonic nebulizer, as is known to those of skill in the art. See e.g., U.S. Pat. No. 4,501,729. Aerosols of solid particles comprising the agents of this invention may likewise be produced with any solid particulate medicament aerosol generator, by techniques known in the pharmaceutical art.

In some embodiments, the agents of the present invention may be delivered via an enteral, parenteral, intrathecal, intracisternal, intracerebral, intraventricular, intranasal, intra-aural, intra-ocular, peri-ocular, intrarectal, intramuscular, intraperitoneal, intravenous, oral, sublingual, subcutaneous and/or transdermal route. In some embodiments, the agents of the present invention may be delivered intracranially and/or intraspinally.

In particular embodiments, the agent of this invention is administered in a liquid formulation by direct injection (e.g., stereotactic injection) to the desired region or compartment. In other embodiments, the agent may be provided by topical application to the desired region or by intra-nasal administration of an aerosol formulation. Administration to the eye may be by topical application of liquid droplets. As a further alternative, the agent may be administered as a solid, slow-release formulation (see, e.g., U.S. Pat. No. 7,201,898).

The following examples are given for the purpose of illustrating various embodiments of the invention and are not meant to limit the present invention in any fashion.

### EXAMPLES

### Example 1. MERS-CoV Manipulates the Unfolded Protein Response to Determine Primary Human Lung Cell Fate

Highly pathogenic human coronaviruses have been observed to cause programmed cell death within in vitro and in vivo models of viral pathogenesis as well as in patient autopsy tissues. Given that MERS-CoV replicates efficiently in multiple cell types essential for lung physiology (e.g., lung fibroblasts, epithelial and endothelial cells), primary culture systems provide a biologically relevant platform to better understand virus and host interactions that may drive severe lung pathology.

MERS-CoV causes severe lung disease but the underlying mechanisms of pathogenesis remain unknown. While much has been learned from the few reported autopsy cases, an in-depth understanding of the cells targeted by MERS-CoV in the human lung and their relative contribution to pathogenesis is needed. To address this knowledge gap, we infected primary human lung microvascular endothelial (MVE) cells and fibroblasts (FB) with MERS-CoV and evaluated the host response over time using a systems level multi-omics approach which revealed that MERS-CoV infected MVE die via apoptotic mechanisms downstream of the unfolded protein response (UPR). Pharmacologic manipulation of the UPR in MERS-CoV infected primary cells and mice confirmed its importance in virus replication and infection outcomes. These data suggest that infection and death of MVE, loss of endothelial barrier integrity and vascular leakage into the airway could be a potential trigger for acute respiratory distress syndrome commonly seen in MERS-CoV patients.

Here we report a kinetic multi-omics analysis of MERS-CoV infected primary human lung fibroblasts (FB) and microvascular endothelial (MVE) cells from three different human donors. While MERS-CoV replication kinetics were similar among FB and MVE, the host responses as determined by transcriptomic, proteomic, and lipidomic analyses differed significantly by cell type and suggested that MVE cells were dying via apoptotic mechanisms initiated by the unfolded protein response (UPR), which helps monitor and maintain homeostasis in the endoplasmic reticulum (ER) following a stress response. Pharmacologic manipulation of UPR demonstrated that active/dimerized protein kinase RNA-like endoplasmic reticulum kinase (PERK) was critical for viral replication in vitro. Of note, mice infected with MERS-CoV and treated with an inhibitor specific for PERK have reduced weight loss and improved respiratory functions, validating that regulation of the UPR influences disease phenotypes in an animal model of lethal MERS-CoV infection. Our studies used analysis of multi-omics datasets derived from MERS-CoV infected primary human lung cells to identify critical host pathways that regulate viral replication and cell survival outcomes and suggest that apoptosis of MERS-CoV infected MVE cells is mediated by the UPR and may represent one of the first breaches of the epithelial-endothelial cell barrier which may allow disease progression to ARDS.

Middle East respiratory syndrome coronavirus (MERS-CoV) causes severe atypical pneumonia with a mortality rate of ˜35%. MERS-CoV infects a variety of primary human lung cell types (fibroblasts, epithelial and endothelial) but their potential contribution to pathogenesis remains unknown. MERS-CoV replicates to similarly high titers in primary human lung microvascular endothelial cells (MVE) and fibroblasts (FB) but significant cytopathic effect and cell death was only observed in MVE. To explore potential mechanisms leading to reduced MVE viability, we infected three sets of donor-matched human lung MVE and FB with MERS-CoV and collected samples for transcriptomic, proteomic, and lipidomic analysis over time which suggested that MERS-CoV-infected MVE were dying via an unfolded protein response (UPR) mediated apoptotic mechanism. MERS-CoV infected MVE, but not infected FB, activated death effector caspases validating that cell death occurs via apoptotic pathways. Treatment of MERS-CoV infected MVE and FB with an inhibitor of the UPR results in decreased levels of replication and treatment of MERS-CoV infected mice with the same inhibitor reduces weight loss and improves respiratory functions validating the critical role the UPR plays in MERS-CoV replication and pathogenesis. These studies suggest that MERS-CoV prevents UPR-mediated homeostasis in the endoplasmic reticulum in infected MVE resulting in apoptotic cell death. Our systems biology approach provides new insight into how MERS-CoV infection of cells essential for lung structure and function may drive severe lung disease in humans while simultaneously identifying novel host targets for therapeutic treatments.

MERS-CoV infection causes cell death in primary human lung endothelial cells but not in primary human lung fibroblasts. The study of MERS-CoV and host interactions in various primary lung cells essential for architecture and physiology provides a unique opportunity to better understand the contributions of individual cell types to disease phenotypes in the lung circumventing one of the biggest challenges in understanding MERS-CoV pathogenicity, a lack of available clinical samples for analysis. To characterize MERS-CoV infection in primary human lung microvascular endothelial cells (MVE) and primary human lung fibroblasts (FB), we obtained virologic and multi-omics data (transcriptomic, proteomic, and lipidomic) from infected cells from three matched human donors every 12 hours for 48 hours. Viral growth kinetics and peak titers (˜10⁷ plaque forming units (PFU)/mL) were nearly identical regardless of donor background. We also determined infection frequency via microscopy by comparing the total number of cells (i.e., nuclei) to the number of MERS-CoV infected cells per high powered field. To simplify this process, we utilized a MERS-CoV expressing the red fluorescent protein (MERS-RFP). Unlike infection frequency in FB, which increased over time, the numbers of total cells per field, as well infection frequency, decreased over time in MVE. Since the number of cells per field in mock-infected MVE cultures did not diminish over time, this suggested that a virus induced cytopathic effect (CPE) was mediating the loss of cells in MERS-CoV infected MVE. To quantitate CPE in MERS-CoV infected MVE and FB over time, we performed a cell viability assay (CellTiter-Glo), which estimates the number of metabolically active viable cells by measuring the levels of adenosine triphosphate (ATP) in each well. Unlike FB, which had similar levels of cell viability in mock- and MERS-CoV-infected cultures for most time points, infected MVE viability decreased significantly over time as compared to mock. Together, these data demonstrate that both primary MVE and FB are similarly susceptible to MERS-CoV infection but MVE cell viability is significantly reduced over time yet FB are relatively unaffected.

Cross-donor consensus transcriptomic, proteomic and lipidomic responses. To gain insight into the divergent outcomes in cell viability following MERS-CoV infection of FB and MVE, we analyzed donor matched kinetic multi-omic datasets. Functional enrichment was performed on transcriptomic and proteomic data across both cell types, which showed distinct differences in the modulation of the transcriptome and proteome among infected FB and MVE. We found that pathways related to apoptosis and the unfolded protein response (UPR) were significantly enriched in MERS-CoV infected MVE at multiple time points, which was not observed in similarly infected FB. Interestingly, increased hemoglobin protein expression levels in both FB and MVE were detected at 48 h pi, which has been demonstrated to regulate endothelial and epithelial cell communication as well as to regulate the durability of vascular walls. We then investigated if mothers against decapentaplegic homolog 7 (SMAD7) and/or fibroblast growth factor 2 (FGF2) transcripts were differentially expressed in MERS-CoV infected MVE or FB as previous studies indicated that upregulated expression of SMAD7 and FGF2 in MERS-CoV infected epithelial cells led to cellular apoptosis. Neither SMAD7 nor FGF2 expression levels were distinct following infection and did not appear to explain the differences in cytopathic effect seen in some studies. Finally, we sought to determine if MERS-CoV infection would alter the lipidome in FB or MVE. We classified lipids into broad categories for enrichment analysis, which revealed differential expression of ceramides and triglycerides in FB and MVE. When analyzed at a per-lipid-species level, we found dramatic and significant differences in specific species of ceramides and triglycerides, both of which are involved in activating apoptotic processes.

MERS-CoV activates the death effector caspases 3 and 7 in infected MVE but not FB. To validate the complementary multi-omics observations suggesting that MERS-CoV infection induces apoptosis in MVE, we simultaneously measured death effector caspases 3 and 7 and cell viability in mock or MERS-CoV infected FB and MVE using the Promega Apotox Triplex kit. We included staurosporine (induces caspase 3/7 activation) or ionomycin (induces necrotic cell death with no caspase activation) treatment as controls. In addition, we added UV-inactivated MERS-CoV virions in order to control for effects on cultures independent of virus replication (i.e., entry, uncoating, etc.). Uninfected MVE and FB treated with staurosporine significantly induced caspase 3/7 activation as compared to mock and cells treated with UV-inactivated MERS-CoV. As expected, ionomycin treatment did not induce caspase 3/7 activation. With MERS-CoV infection, significant caspase 3/7 activation was observed at both 24 and 48 hours post infection but only in MVE and not in FB. Caspase activation was not observed with UV-inactivated MERS-CoV treatment or in mock-infected cells for either cell type. When measuring cell viability within the same assay, neither mock infection nor treatment with UV-inactivated appreciably affected cell viability. Importantly, treatment of both FB and MVE with control compounds (e.g., staurosporine or ionomycin) diminished cell viability but MERS-CoV infection only diminished cell viability in MVE. Death of MERS-CoV infected MVE could lead to permeability at the epithelial/endothelial cell barrier as one of the first steps of ARDS and viable MERS-CoV infected FB would continue to produce high levels of infectious virus, both of which would contribute to the overall disease burden in the infected patient.

Differential transcriptomic and proteomic signatures for UPR chaperones in MERS-CoV infected MVE and FB. Both systems data and confirmatory experimental validation studies suggested that MERS-CoV infected MVE were dying via apoptotic pathways. To better understand the initiation of this process, we re-analyzed our systems biology data focusing on specific mediators of apoptosis and UPR. We found increased protein expression of three well-established UPR markers, glucose regulated protein 78 (GRP78), heat shock protein 90 kDa beta member 1 (HSP90B1) and calnexin (CANX) in MERS-CoV infected MVE but not infected FB. GRP78 is a master regulatory protein for the UPR that binds to regulatory enzymes within the UPR pathway, keeping them inactive unless misfolded proteins accumulate, triggering dissociation and enzyme activation. HSP90B1 and CANX are endoplasmic reticulum (ER) protein chaperones that facilitate nascent protein folding. Interestingly, transcripts levels for all three UPR markers were strongly down-regulated in both MERS-CoV infected MVE and FB. The discrepancy between transcripts and protein may indicate conflicting regulatory signals as the virus establishes control and establishes replication complexes and begins to assemble progeny virions in the midst of the host response to the invasion. Cumulatively, these data suggest that MERS-CoV induction of the UPR is specific to a particular cellular environment (MVE) and may not be broadly applicable to all infected cells and tissues.

Validation studies to perturb the UPR in MERS-CoV infected MVE and FB suggest PERK activation is critical for viral replication. To further validate the results thus far, which suggested that apoptosis of MVE was associated with the UPR, we pharmacologically perturbed the UPR in MERS-CoV infected MVE and FB using AMG44, an inhibitor specific to the first enzyme activated following misfolded protein accumulation, protein kinase R-like endoplasmic reticulum kinase (PERK). Activated PERK regulates phosphorylation of the key translation mediator, elongation initiation factor 2α (eIF2α). Following activation of the UPR and PERK, host translational shutoff serves to reestablish ER homeostasis and proper protein folding, which if not achieved, results in the activation of apoptotic pathways.

Dose response studies were performed in mock and MERS-CoV infected MVE and FB with a PERK inhibitor (AMG 44 at 100 to 2 μM) to determine if pharmacologic perturbation of the PERK arm of the UPR would affect MERS-CoV replication and/or cell death. We utilized a MERS-CoV nanoluciferase reporter virus (MERS nanoluc) to increase accuracy and throughput of the assay. PERK inhibition diminished MERS-CoV replication in both FB and MVE in a dose dependent manner when assayed at either 24 or 48 hours post infection. In addition to monitoring changes in virus replication in the context of PERK inhibition, we also monitored cytotoxicity in drug treated uninfected cultures via CellTiter-Glo cell viability assay. Unlike FB in which we did not observe cytotoxicity at either time point, in MVE we observed a dose dependent increase in cytotoxicity with PERK inhibition that was most notable at 48 h pi. Importantly, we found that at 24 h pi, MERS-CoV replication was inhibited at multiple drug doses that did not exhibit cytotoxicity. These results suggest that primary human lung MVE are more sensitive to inhibition of the UPR in the absence of infection in contrast to the more tolerant primary lung FB.

To determine if PERK inhibition modifies induction of apoptosis following MERS-CoV infection, we performed similar dose response assays, but then assayed for caspase 3 and 7 activation using similar UV inactivated and small molecule controls (i.e., staurosporin, ionomycin). No significant dose responsive change in caspase 3/7 activation above untreated infected cells was detected in either primary human lung MVE or FB following MERS-CoV infection and treatment with PERK inhibitor.

PERK inhibition diminishes MERS-CoV pathogenesis and improves pulmonary function. Because in vitro PERK inhibition resulted in a significant decrease in MERS-CoV replication in both cell types, we sought to determine if in vivo inhibition would result in a change in viral replication and/or pathogenesis. To address this, we utilized a transgenic mouse model where the murine ortholog of the human MERS-CoV receptor, dipeptidyl peptidase 4 (DPP4), was humanized at residues 288 and 330 (hDPP4), facilitating high titer replication localized to the respiratory tract and lung pathology similar to that observed in humans. We initiated treatment of hDPP4 mice with AMG44 or vehicle once per treatment at 24 hours prior to and post MERS-CoV infection. By three days post MERS-CoV infection, vehicle treated mice lost significantly (p value <0.05) more weight than AMG44 treated mice and this difference was maintained for the remainder of the study. Interestingly, virus titer in the lung on day 3 post infection was similar regardless of treatment. “Pulmonary hemorrhage,” a gross pathological phenotype that increases coincident with MERS-CoV pathogenesis, was significantly diminished in AMG44 treated mice as compared to those receiving vehicle treatment. To determine if PERK inhibition through AMG44 treatment would affect pulmonary function, we performed whole body plethysmography (WBP) daily. Concordant with pulmonary hemorrhage, AMG44 treatment improved pulmonary function as compared to those receiving vehicle. For three WPB metrics typically induced following MERS-CoV infection, Pause, Penh and EF50, AMG44 treated mice had significantly lower values (p value <0.05) than vehicle treated animals at multiple time points. Pause, PenH and EF50 are all measures of airway constriction or obstruction and indicate that AMG44 treatment is relieving or preventing lung pathology that negatively affects lung function. Together with the wide variety of supportive data described above, the in vivo studies with AMG44 directly demonstrate the importance of PERK signaling in promoting MERS-CoV pathogenesis. Thus, with our unbiased systems biology-based approach, we identified a pathway modulated during MERS-CoV infection of primary human cells, validated the importance of this pathway for MERS-CoV replication and cell death in vitro and then demonstrated its importance in viral pathogenesis, thus confirming the utility of this approach for identifying host factors/pathways that could be targeted as antiviral therapy.

Primary human lune cells and immortalized cells. Primary human lung cells were isolated from distal lung tissue and processed as described. Human lung microvascular endothelial cells (MVE) were cultured in Vasculife VEGF-MVE Endothelial Media (Lifeline Cell Tech). Human lung fibroblasts (FB) were cultured in DMEM-H Basal Medium (Corning), 1× penicillin/streptomycin (Sigma), 10% fetal bovine serum (Gemini Bio). Media fetal bovine serum concentrations were reduced to 4% prior to infection. Vero 81 cells were cultured in Dulbecco's modified essential media (DMEM Gibco) with 10% fetal clone II (Hyclone) and 1× antibiotic/antimycotic (Gibco).

Viruses and viral titration. Wild-type MERS-CoV (EMC 2012 strain), MERS-CoV expressing nano luciferase (MERS nanoluc) and MERS-CoV expressing the red fluorescent protein (MERS-RFP) were rescued from infectious clones. Vero C81 cells were used to generate viral stocks and to quantitate viral titers following infection.

Infections to collect transcriptomic, proteomic, and lipidomic samples. Donor matched MVE and FB from three different human tissue donors were infected and samples were harvested for systems biology analysis. For transcriptomic analysis, five replicate wells of MVE or FB were either mock-infected or infected with wild type MERS-CoV at a multiplicity of infection (MOI) of 5. At 0, 12, 24, 36, and 48 hours post infection (pi), 100 uL of media/supernatant was collected for viral titration assays, media removed and then 1 mL of Trizol (Invitrogen/ThermoFisher) added per well. Total RNA samples were sent to Arraystar for downstream analysis. In parallel, an additional five replicate wells were subjected to chloroform/methanol (2:1 mix) precipitation. All proteomic and lipidomic samples were desiccated and frozen prior to shipment to Pacific Northwest National Laboratories for downstream analysis.

Transcriptomic analysis. Scanned images were analyzed using Agilent Feature Extraction Software (v11.0.1.1). The limma package for R (available on Bioconductor) was used to perform background correction, quantile normalization (normalizeBetweenArrays), and summarization (avereps) to derive a single normalized intensity value per probe. Outlier samples were detected using PCA and by visual inspection of heatmaps, and all data was re-processed after removing outlier samples. All data processing for each of the biological replicates was performed independently of the other.

Proteomic analysis. The algorithm RMD-PAV was used to identify any outlier biological samples, which was confirmed via Pearson correlation. Peptides with inadequate data for either qualitative or quantitative statistical tests were also removed from the dataset. The SPANS algorithm was used to identify the best normalization method for each dataset. Peptides were evaluated with Analysis of Variance (ANOVA) with a Dunnett test correction and a Bonferroni-corrected g-test to compare each virus to the associated mock within each time point. To perform signature-based protein quantification, BP-Quant, each peptide was categorized as a vector of length equal to the number of viruses being evaluated. If all comparisons for all time points are 0 for a specific virus it is considered as non-changing and given a value of 0. If there are more time points with an increase in virus to mock than decreasing it is categorized as a +1 and the contrary −1 is given for the decrease in virus to mock. BP-Quant was run with a default parameter of 0.9. All proteins were then analyzed using the same methodology as for the peptides; ANOVA with a Dunnett test correction and a Bonferroni-corrected g-test to compare each virus to the associated mock within each time point.

Lipidomic analysis. The algorithm RMD-PAV was used to identify any outlier biological samples, which was confirmed via Pearson correlation Lipids with inadequate data for either qualitative or quantitative statistical tests were also removed from the dataset. Median centering was used for normalization. Lipids were evaluated with a standard two sample t-test to compare each infected condition to the associated mock within each time point.

Functional enrichment. We identified significantly changed features (transcripts, proteins, lipids) by using an adjusted p-value of 0.05 as a threshold. For each experimental condition, perturbed features were divided into one list each of up- and down-regulated items. Each list was tested against the Gene Ontology (GO) database of gene sets using the EASE-adjusted one-sided Fisher exact test, such that each condition is tested for up- and down-regulation of each function/pathway in the ontology database. Results across multiple conditions were visualized in heat maps using the log 10 p-values of the significance tests, colored to indicate the direction of change. For lipids, feature sets were generated by categorizing the lipids observed in our experiments according to broad lipid classifications; these classifications were then used as “gene sets” and the enrichment analysis was then performed in the same manner as for transcripts and proteins.

Cell viability and percent infection assays. To assess numbers of viable cells following MERS-CoV infection of FB and MVE, we used the CellTiter-Glo Luminescent Cell Viability Assay (Promega) at 24 and 48 hours post infection/treatment according to manufacturer's instructions and relative light units were determined by Spectromax plate reader (Molecular Devices), averaged and plotted and statistical significance between mock and infected wells determined by Manfield Whitney unpaired t test (performed in GraphPad Prism). Percent infection was determined using MVE and FB infected with MERS-RFP (MOI 5) or mock-infected and counting nuclei (Horcht stain diluted 1:5,000 in media) in replicate wells at 0, 12, 24, 36, and 48 hours post infection. Cell numbers were determined using the Fiji cell counter function and total number of cells versus number of red fluorescent cells were determined for at least three wells per condition per cell type. Statistical significance was determined by Manfield Whitney unpaired t test (performed in GraphPad Prism).

Apoptosis assays. Infected FB and MVE were assayed using either the Caspase-Glo 3/7 Kit (Promega) to measure caspase activation or using the ApoTox-Glo Triplex Assay (Promega) to assess cell viability, cytotoxicity and caspase 3/7 activation in a single set of samples at 24 and 48 hours post infection according to manufacturer's instructions. Control wells were treated with either staurosporin (Sigma, 8 uM for MVE or 10 uM for FB) or ionomycin (ThermoFisher, 40 uM for MVE and 60 uM for FB). Relative light units or emission excitation wavelengths were determined by Spectromax plate reader (Molecular Devices). Statistical significance was determined by Manfield Whitney unpaired t test (performed in GraphPad Prism).

Inhibitor studies. MERS-CoV infected MVE and FB were tested at 24 and 48 hours post infection following treatment with a dilution series PERK inhibitor (AMG 44{Rojas-Rivera, 2017 #74; Smith, 2015 #73} 100 μM to 2 μM) and was evaluated for cytotoxicity with drug treatment alone (cell viability assay, no virus), activation of the death caspases 3/7 (wild type MERS-CoV) and viral replication (detection of luciferase as a surrogate for replication, MERS nanoluc virus). All assay results were read by SpectraMax (Molecular Devices). Results are graphed as percent inhibition and toxicity (effective and cytotoxic concentration 50) as determined by GraphPad Prism.

Mouse studies. 288-330^{+/+} mice were housed and bred in accordance with the University of North Carolina Department of Comparative Medicine, AALAC #329. For a single group of mice, dosing occurred once a day 24 hours prior to infection and a second single dose was administered 24 hours post infection via the intraperitoneal route (12 mg/kg of PERK AMG44 suspended in water or with a sham control). Immediately prior to infection, mice were anesthetized with 50 μL of ketamine/xylazine mixture via intraperitoneal injection. 16-20 week old mice were intranasally infected with 10⁴ PFU of mouse adapted MERS-CoV maM35c4 diluted in OPTIMEM, a total volume of 50 μL was given. Mice were weighed daily following infection and had their respiratory function measured one day prior to infection and at days two through six post infection using Buxco Whole Body Plethysmography (Data Sciences International). Briefly, mice were allowed to acclimate for 30 minutes in the plethysmography chamber located within the biological safety cabinet after which a 5 minute measurement was taken. Readings were collected every 2 seconds for a total of 150 measurements per mouse per day. At days three and seven post infection mice were euthanized via isoflurane overdose after which lung tissue was harvested to determine viral replication titers. Gross pulmonary hemorrhage was observed and scored from zero (none) to four (severe and total) at the time of dissection.

### Example 2. Unfolded Protein Response Inhibition Reduces Middle East Respiratory Syndrome Coronavirus Induced Acute Lung Injury

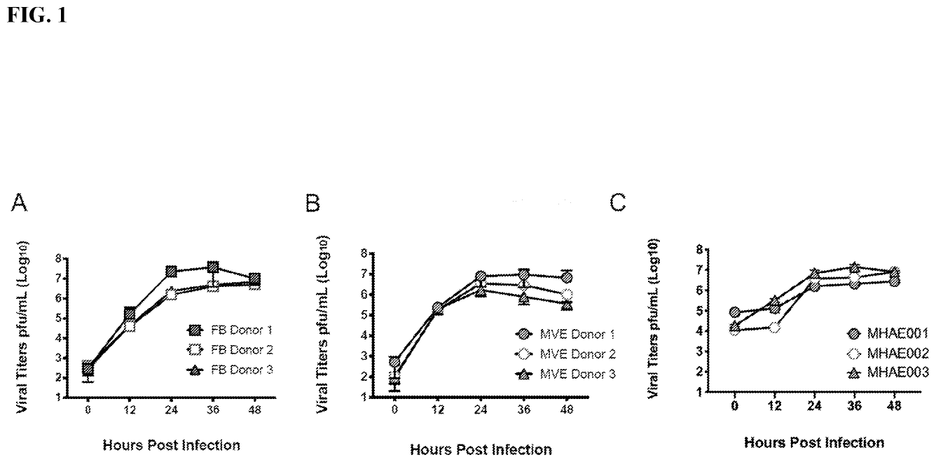

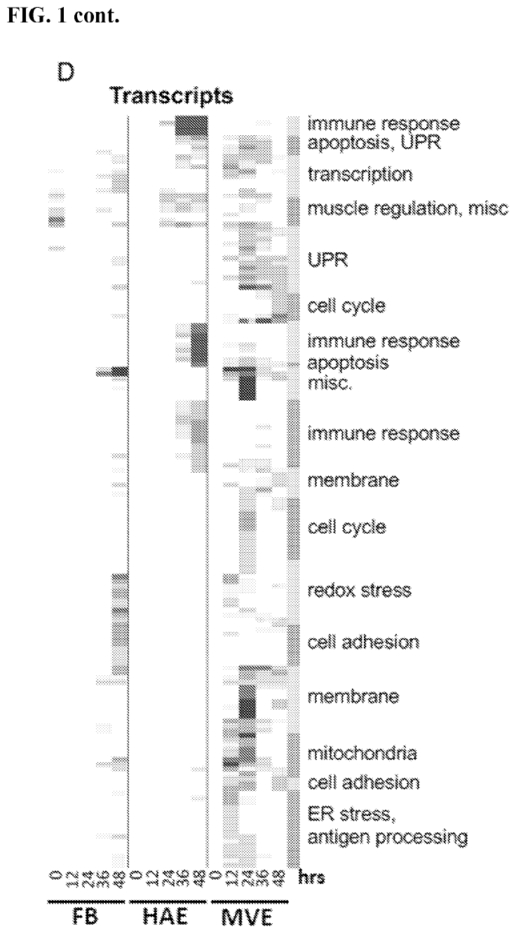

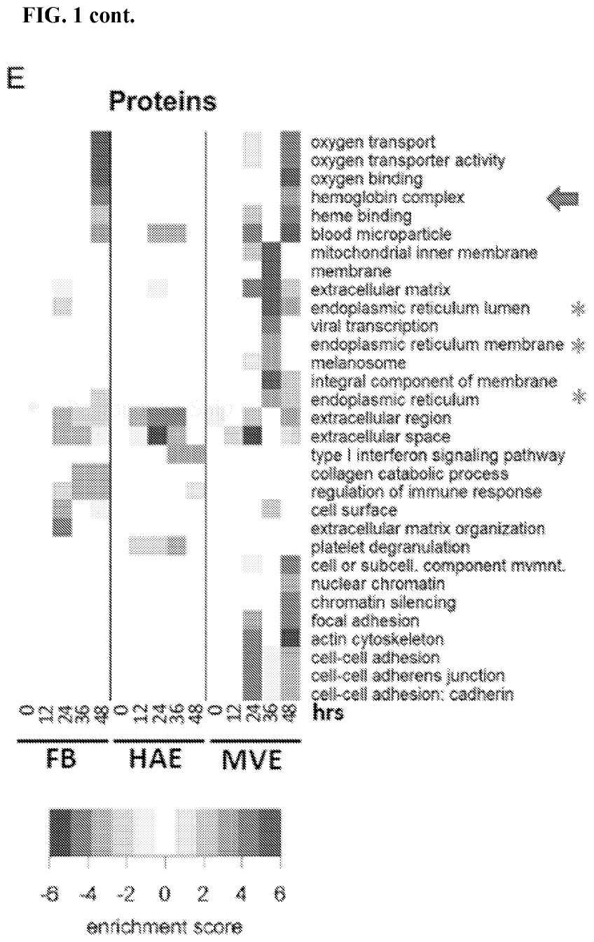

Identifying MERS-CoV pathogenic mechanisms requires understanding critical virus-host interactions in infected primary human lung cells that provide the vital structural and physiological requirements for lung function. As clinical samples for MERS-CoV are scarce, using matched primary human lung cells from previously healthy donors provides a novel opportunity to elucidate tissue specific changes that may explain disease phenotypes. To characterize MERS-CoV infection in primary human lung airway epithelial cell cultures (HAE), primary human lung microvascular endothelial cells (MVE) and primary human lung fibroblasts (FB), we infected cells from three matched human donors at a multiplicity of infection of 5 (MOI 5) and conducted virologic and multi-omic (transcriptomic, proteomic, and lipidomic) analyses at 0, 12, 24, 36 and 48 hours post infection. Interestingly, viral growth kinetics and peak titers (˜10⁷ plaque forming units (PFU)/mL) were nearly identical regardless of donor background (FIG. 1 panels A-C), with a slight expected delay in viral replication kinetics in HAE cultures. To gain insight into the host pathways that were uniquely or commonly differentially regulated by MERS-CoV infection of HAE, FB and MVE, functional enrichment was performed on donor matched kinetic transcriptomic and proteomic data across all three cell types, using an approach where only the least significant response of the three donors is preserved for each ontology term. This conservative enrichment strategy ensures that only responses that are most likely to be conserved across the human population are displayed. This study found distinct differences in the modulation of the transcriptome and proteome among infected HAE, FB and MVE (FIG. 1 panels D and E), revealing pathways related to apoptosis (transcription, FIG. 1 panel D) and the unfolded protein response (UPR, transcripts and proteins, FIG. 1 panels D and E), were significantly enriched in MERS-CoV infected MVE at multiple timepoints, but not observed in similarly infected HAE or FB. Interestingly, immune-related responses were detected only in HAE transcriptomics, suggesting that this is the only cell type of the three that programs an innate immune response and suggests a marked vulnerability of lung FB and MVE to infection. Susceptibility of MVE and FB to MERS-CoV infection may also contribute to the higher mortality rates for MERS-CoV patients compared to SARS-CoV 1 & 2 infected individuals, since the latter viruses do not infect FB and MVE. Increased hemoglobin protein expression levels in FB and MVE at 48 hours post infection were also observed (arrow, FIG. 1 panel E), which has been demonstrated to regulate endothelial and epithelial cell communication as well as to regulate the durability of vascular walls. This increase was not detected in infected HAE.

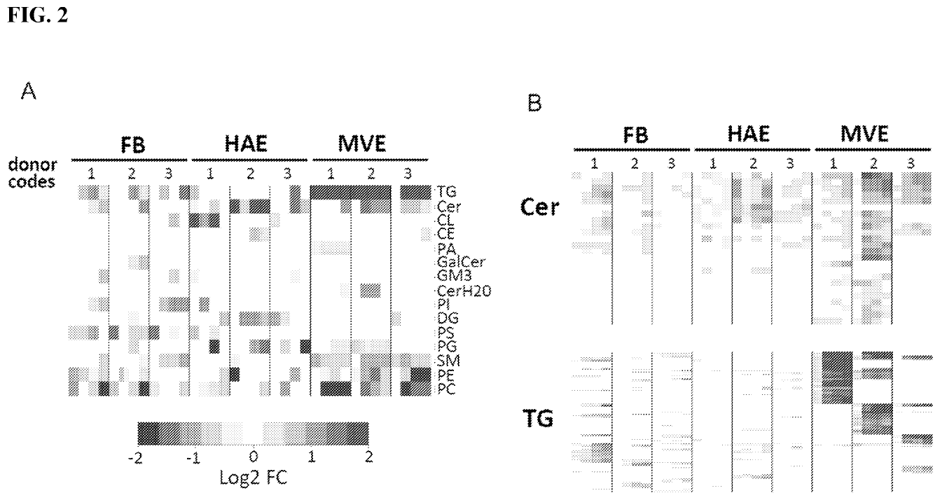

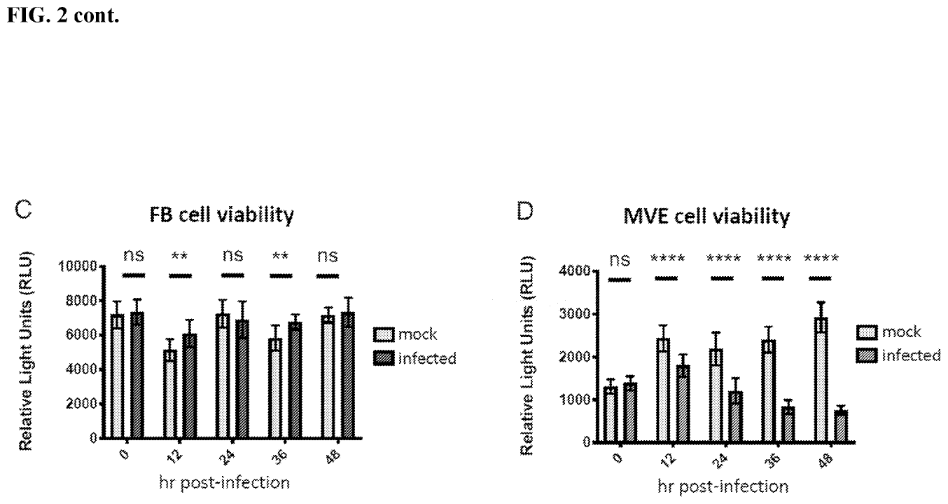

This study also sought to determine if MERS-CoV infection alters the lipidome in HAE, FB and/or MVE. Lipids were classified into broad categories for enrichment analysis, which revealed differential expression of ceramides and triglycerides in HAE, FB and MVE (FIG. 2 panel A). When analyzed at a per-lipid-species level, significant differences were found in specific species of ceramides (FIG. 2 panel B, upper panel) and triglycerides (FIG. 2 panel B, lower panel) in MERS-CoV infected MVE, both of which are involved in activating apoptotic processes. Given their apparent immune vulnerability and the likely contrast in apoptotic/UPR pathway response between FB and MVE, this study on these two cell types.

MERS-CoV induces cytopathic effect in primary human lune endothelial cells but not fibroblasts. To determine how the differences observed from omics studies affected cell viability when the cell types were compared, microscopy studies were performed to determine total numbers of mock-infected and MERS-CoV-infected MVE and FB per field at each timepoint. For both mock-infected and infected FB the total numbers of cells increased through 36 hours post infection with little difference between mock-infected or infected cell counts at any time post infection. In contrast, while the number of mock-infected MVE was steady over the course of the infection, numbers of infected MVE significantly declined over time. Since the number of cells per field in mock-infected MVE cultures did not diminish over time, virus-induced cytopathic effect (CPE) was likely mediating the loss of cells in MERS-CoV infected MVE. To quantitate CPE in MERS-CoV infected MVE and FB over time, a cell viability assay (CellTiter-Glo) was performed which estimates the number of metabolically active viable cells by measuring the levels of adenosine triphosphate (ATP) in each well. Unlike FB which had similar cell viability estimates in mock-infected and infected cells, infected MVE viability decreased significantly over time as compared to mock (FIG. 2 panels C and D), suggesting an infection induced loss in cell viability. Together, these data demonstrate that MERS-CoV infections in both primary MVE and FB are similarly productive yet differentially regulate distinct host pathways leading to significantly reduced MVE, but not FB, cell viability over time.

This study then investigated if mothers against decapentaplegic homolog 7 (SMAD7) and/or fibroblast growth factor 2 (FGF2) transcripts were differentially expressed in MERS-CoV infected MVE or FB, as previous studies indicated that upregulated expression of SMAD7 and FGF2 in MERS-CoV infected immortalized kidney and lung epithelial cells led to cellular apoptosis. Neither SMAD7 nor FGF2 expression levels were distinct following infection and did not appear to explain the differences in cytopathic effect seen in the current studies.

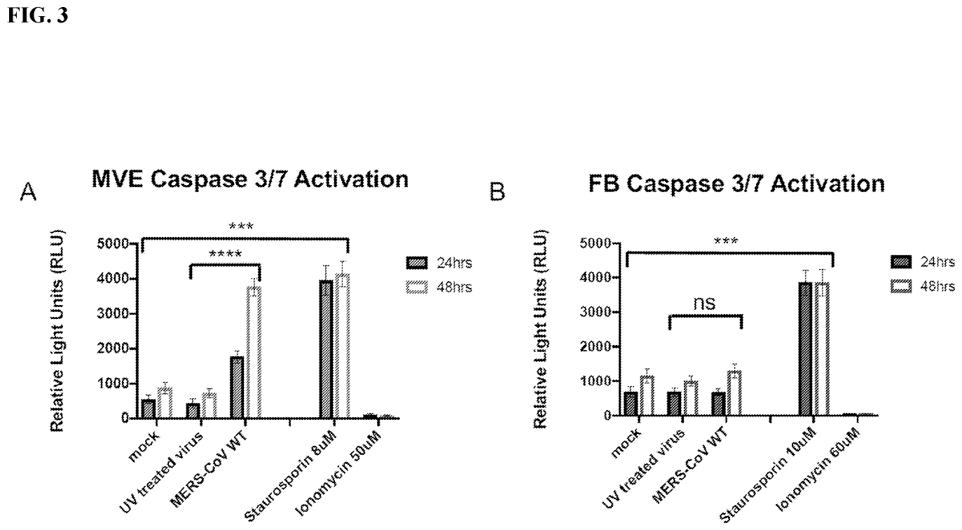

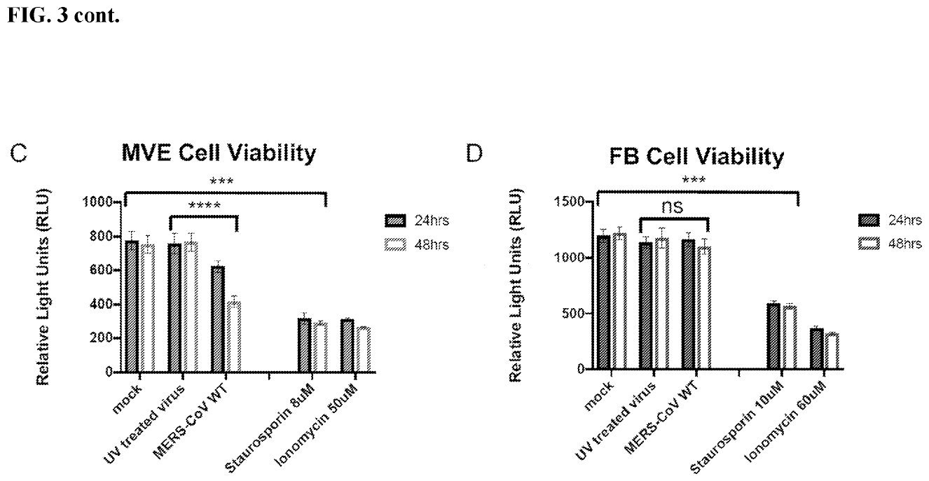

MERS-CoV activates Caspases 3 and 7 in infected MVE. To validate the complementary multi-omics observations suggesting that MERS-CoV infection induces apoptosis in MVE, this study simultaneously measured death effector caspases 3 and 7 and cell viability in mock or MERS-CoV infected FB and MVE using the Promega Apotox Triplex kit. Staurosporine (induces caspase 3/7 activation) or ionomycin (induces necrotic cell death with no caspase activation) treatment were included as controls. In addition, UV-inactivated MERS-CoV virions were added in order to control for effects on cultures independent of virus replication (i.e., entry, uncoating, etc.). Uninfected MVE and FB treated with staurosporine significantly induced caspase 3/7 activation as compared to mock and cells treated with UV-inactivated MERS-CoV (FIG. 3 panels A and B). As expected, ionomycin treatment did not induce caspase 3/7 activation. With MERS-CoV infection, significant caspase 3/7 activation was observed at both 24 and 48 hours post infection, but only in MVE (FIG. 3 panel A) not in FB (FIG. 3 panel B). Caspase activation was not observed with UV-inactivated MERS-CoV treatment or in mock-infected cells for either cell type (FIG. 3 panels A and B). When measuring cell viability within the same assay, neither mock infection nor treatment with UV-inactivated virions appreciably affected cell viability (FIG. 3 panels C and D). Importantly, treatment of both FB and MVE with control compounds (e.g., staurosporine or ionomycin) diminished cell viability, but MERS-CoV infection only diminished cell viability in MVE (FIG. 3 panels C and D). Death of MERS-CoV infected MVE could lead to permeability at the epithelial/endothelial cell barrier as one of the early steps of ARDS while viable MERS-CoV infected FB would continue to produce high levels of infectious virus both of which would contribute to the overall disease burden in the infected patient.

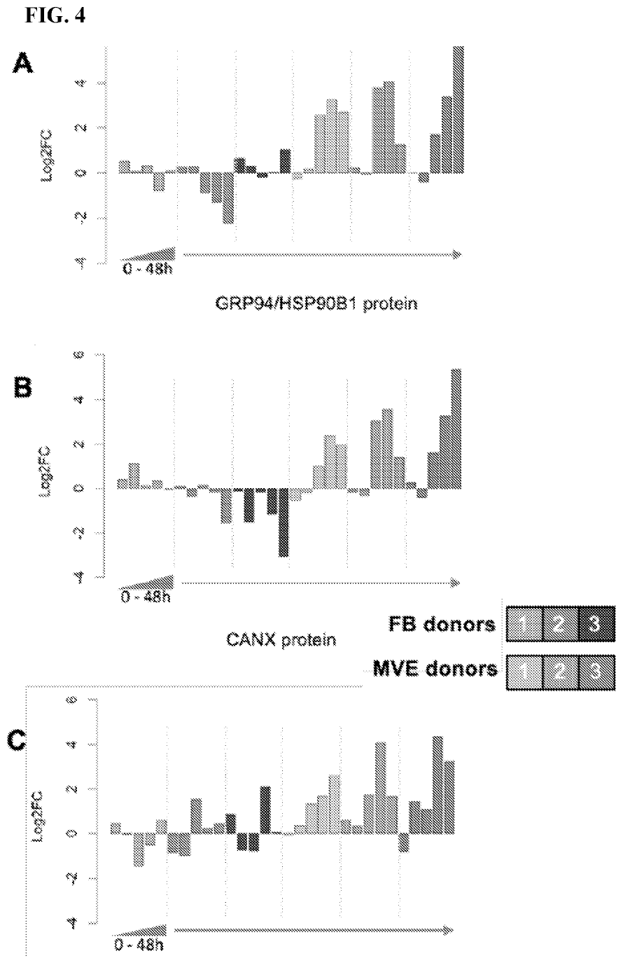

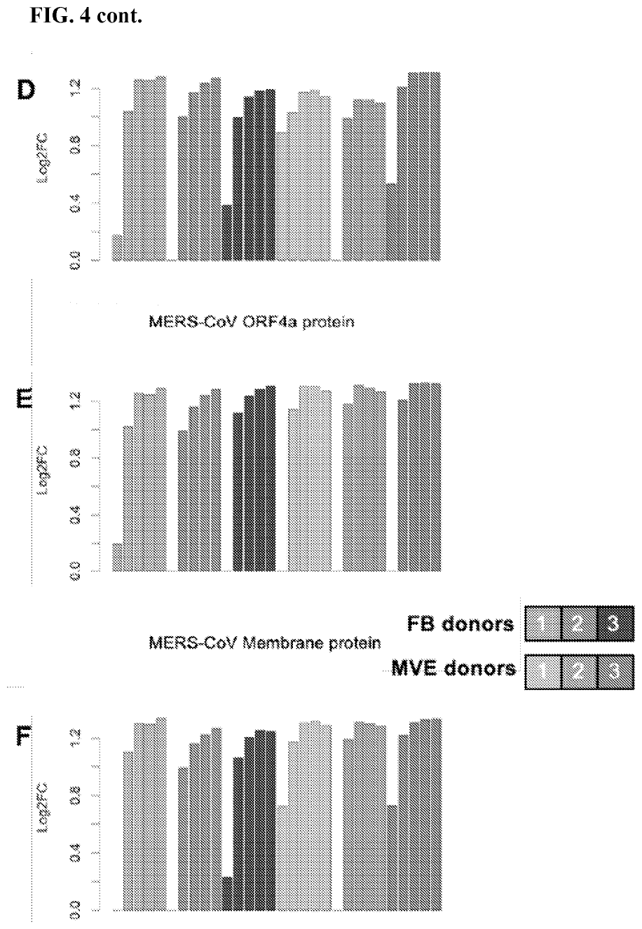

Expression of proteins indicative of the UPR pathway are increased in MERS-CoV infected MVE. Both ‘omics data and confirmatory experimental validation studies suggested that MERS-CoV infected MVE were dying via apoptotic pathways. To better understand the initiation of this process, we re-analyzed our ‘omics data focusing on specific mediators of apoptosis and UPR. We found increased protein expression of three well-established UPR markers, glucose regulated protein 78 (GRP78; also known as binding immunoglobulin protein (BiP)), heat shock protein 90 kDa beta member 1 (HSP90B1; also known as GRP94) and calnexin (CANX) in MERS-CoV infected MVE, but not infected FB (FIG. 4 panels A-C). GRP78/BiP is a master regulatory protein for the UPR that binds to regulatory enzymes within the UPR pathway keeping them inactive unless misfolded proteins accumulate triggering dissociation and enzyme activation. HSP90B1/GRP94 and CANX are ER protein chaperones that facilitate nascent protein folding. In support of the viral titer data, the expression of MERS-CoV structural proteins, spike glycoprotein and membrane protein as well as the accessory open reading frame protein 4a (ORF4a) were expressed to similar levels in both cell types for all donors by 12 hours post infection (FIG. 4 panels D-F). Cumulatively, these data suggest that MERS-CoV modulation of the UPR is specific to a particular cellular environment (MVE) and may not be broadly applicable to all infected cells and tissues.

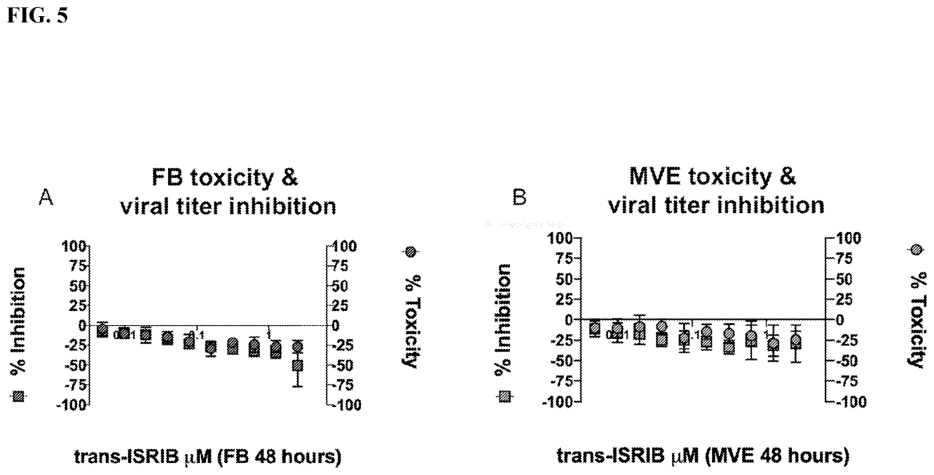

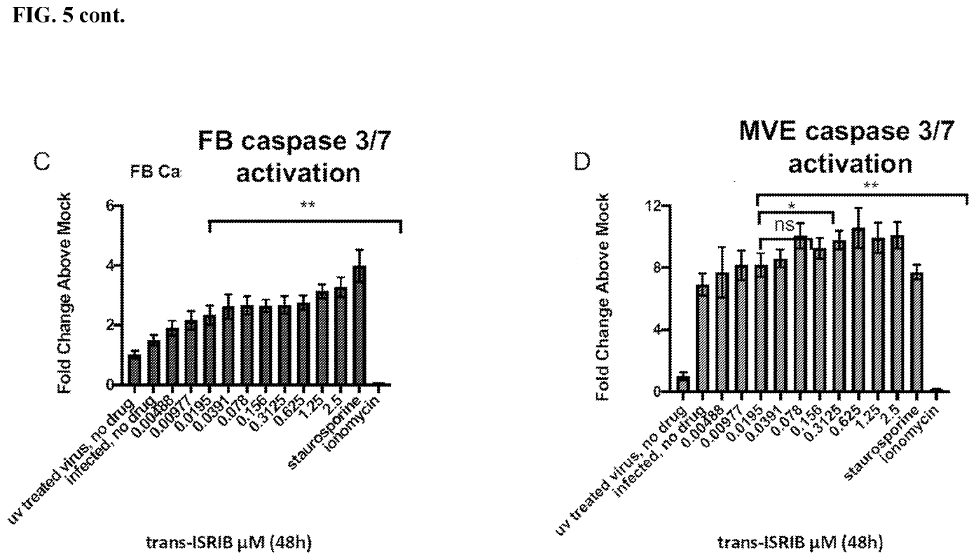

MERS-CoV infection activates stress response pathways in primary human lune MVE and FB. Following external stimuli or pathogen invasion, cells will often halt global cellular translation to attempt to reestablish homeostasis. Cell viability, transcriptomic and proteomic data suggested that MERS-CoV infected MVE but not FB have activated cellular stress response (UPR and apoptosis) pathways. To validate activation of cellular stress response pathways in MERS-CoV infected MVE and FB, lung cells were simultaneously infected and treated with serial doses of the integrated stress response inhibitor, trans-ISRIB. The integrated stress response (ISR) is mediated by one of four kinases (protein kinase R-like endoplasmic reticulum kinase (PERK), heme-regulated inhibitor (HRI), general control non-depressible 2 (GCN2), and double stranded RNA dependent protein kinase (PKR)) that can all phosphorylate the key translation mediator, elongation initiation factor 2alpha (eTF2alpha). Phosphorylated eTF2alpha shuts down global host translation to allow the cell time to recover from a variety of stressful stimuli; however, if recovery is not possible then apoptotic processes are initiated. The inhibitor renders cells no longer sensitive to eTF2alpha phosphorylation. In cells that are not stressed, trans-ISRIB has no effect, but in cells with activated stress pathways (specifically PERK activated ones) treatment with trans-ISRIB results in decreased cell viability. Dose response studies were performed in mock and MERS-CoV infected MVE and FB with trans-ISRIB (2.5 μM to 0.00488 μM) to determine if pharmacologic perturbation of the ISR would affect MERS-CoV replication and/or cell death. A MERS-CoV nanoluciferase reporter virus (MERS nanoluc) was used to increase accuracy and throughput of the assay. Inhibition of the ISR did not alter MERS-CoV replication in either infected MVE or FB (FIG. 5 panels A and B). In addition to monitoring changes in virus replication in the context of inhibition of the ISR, cytotoxicity in drug-treated uninfected cultures was also monitored via CellTiter-Glo cell viability assay. Treatment with trans-ISRIB was not cytotoxic at any dose tested (FIG. 5 panels A and B). To determine if inhibition of the ISR modifies induction of apoptosis following MERS-CoV infection, similar dose response assays were performed as described in FIG. 5 panels A and B but then assayed for caspase 3/7 activation as in FIG. 3 using similar UV inactivated and small molecule controls (i.e., staurosporin, ionomycin). Significant dose-responsive changes in caspase 3/7 activation above untreated infected cells were detected for both primary human lung MVE and FB following treatment with trans-ISRIB suggesting that both cell types activate and benefit from stress response pathways following MERS-CoV infection.

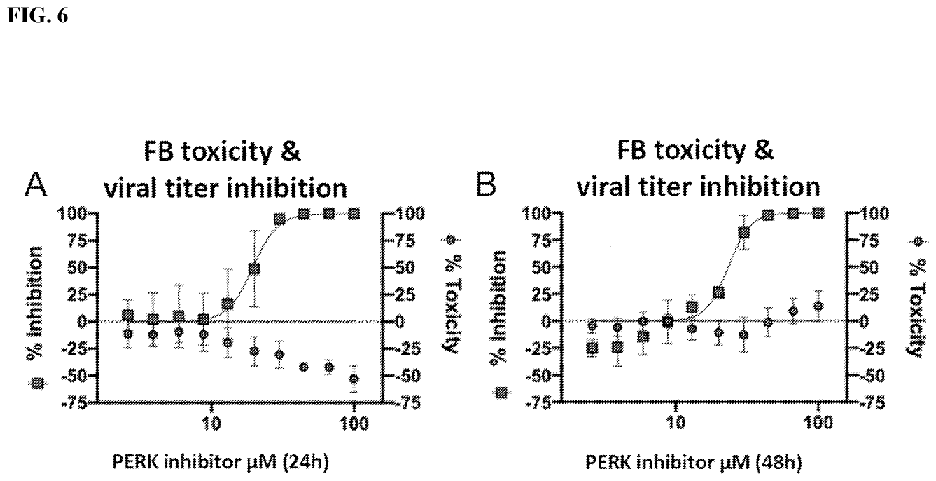

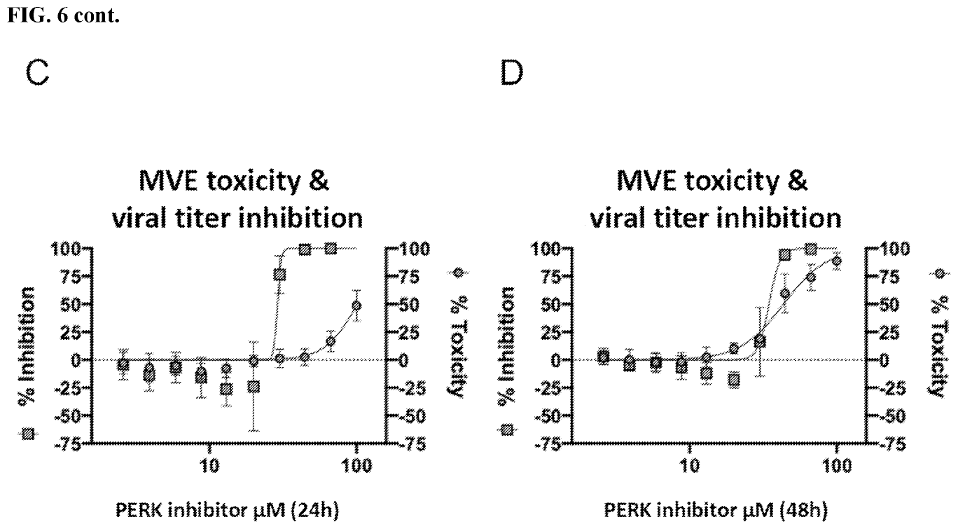

PERK activation is critical for MERS-CoV replication in primary human lune MVE and FB. To further validate the results thus far, which suggested that apoptosis of MVE was associated with the UPR, this study pharmacologically perturbed the UPR in MERS-CoV infected MVE and FB using an inhibitor specific to the first enzyme activated following misfolded protein accumulation, PERK (AMG PERK 44). Activated PERK regulates phosphorylation of eIF2alpha in the UPR. Following activation of the UPR and PERK, host translational shutoff serves to reestablish ER homeostasis and proper protein folding, which if not achieved, results in the activation of apoptotic pathways. Dose response studies were performed in mock and MERS-CoV infected MVE and FB with high doses of PERK inhibitor (AMG PERK 44 at 100 to 2 μM) to determine if pharmacologic perturbation of the PERK arm of the UPR would affect MERS-CoV replication and/or cell death. Inhibition of PERK diminished MERS-CoV replication in both FB (FIG. 6 panels A and B) and MVE (FIG. 6 panels C and D) in a dose-dependent manner when assayed at either 24 (FIG. 6 panels A and C) or 48 hours post infection (FIG. 6 panels B and D) using the same MERS-CoV nanoluc assays described for trans-ISRIB. In addition to monitoring changes in virus replication in the context of PERK inhibition, cytotoxicity in drug treated uninfected cultures was also monitored via CellTiter-Glo cell viability assay. Unlike FB in which we did not observe cytotoxicity at either timepoint (FIG. 6 panels A and B), in MVE we observed a dose dependent increase in cytotoxicity with PERK inhibition that was most notable at 48 hpi (FIG. 6 panel D). Importantly, it was found that at 24 hours post infection, MERS-CoV replication was inhibited at multiple drug doses that did not exhibit cytotoxicity. These results suggest that primary human lung MVE are more sensitive to inhibition of the UPR in the absence of infection in contrast to the more tolerant primary lung FB. To determine if PERK inhibition modifies induction of apoptosis following MERS-CoV infection, similar dose response assays as described in FIG. 6 panels A-D were performed but then assayed for caspase 3/7 activation as in FIG. 3 using similar UV inactivated and small molecule controls (i.e., staurosporin, ionomycin). No significant dose responsive change in caspase 3/7 activation above untreated infected cells was detected in either primary human lung MVE or FB following MERS-CoV infection and treatment with PERK inhibitor. Taken together, the results from trans-ISRIB and AMG PERK 44 treatment suggest that both FB and MVE undergo stress response activation during infection, which is exacerbated when the ability to slow translation through eIF2alpha phosphorylation is removed, and that viral production is dependent on non-eIF2alpha-related signaling through PERK kinase.

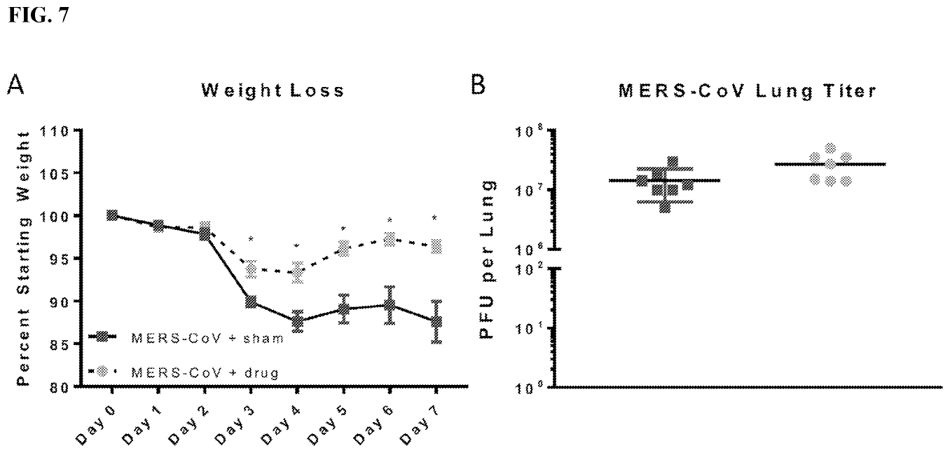

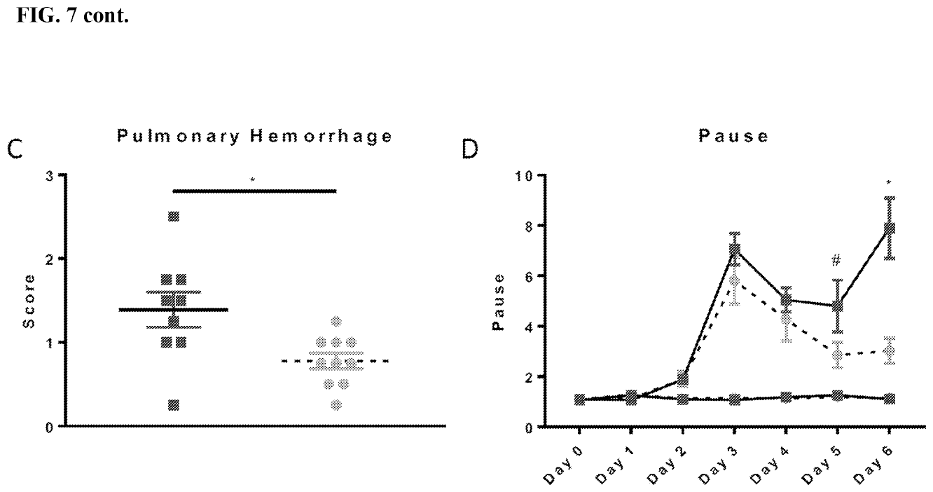

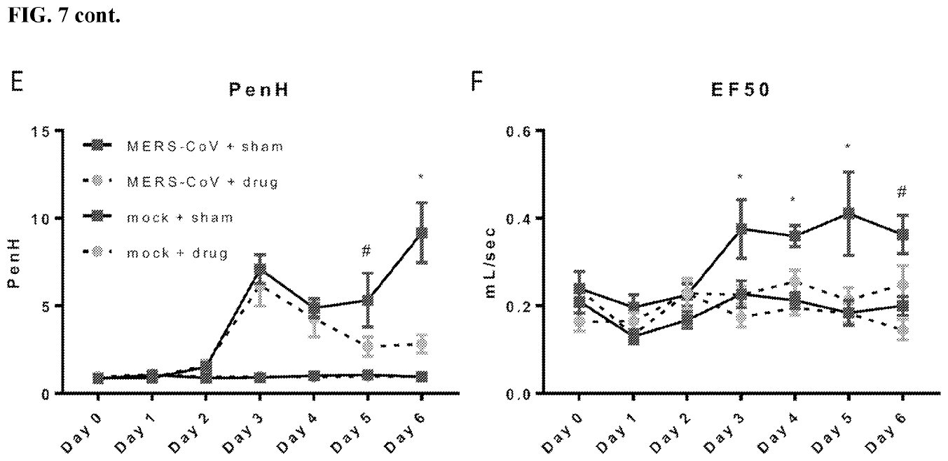

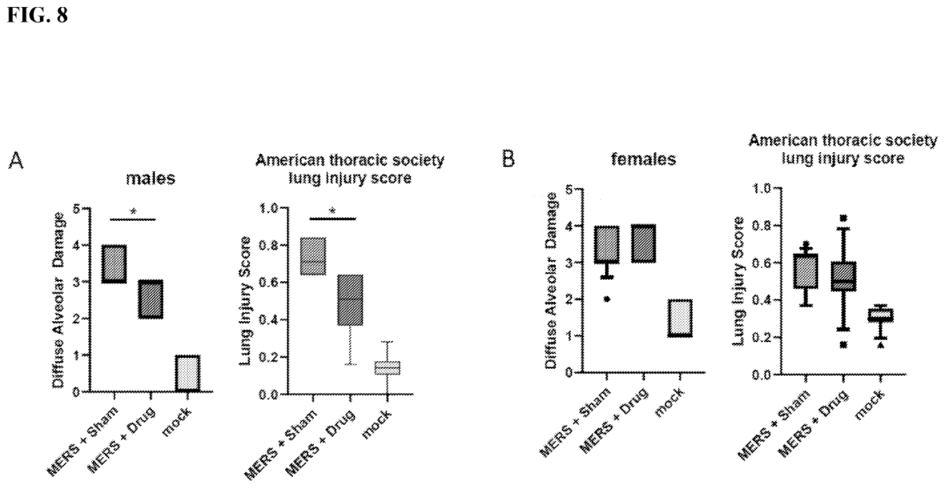

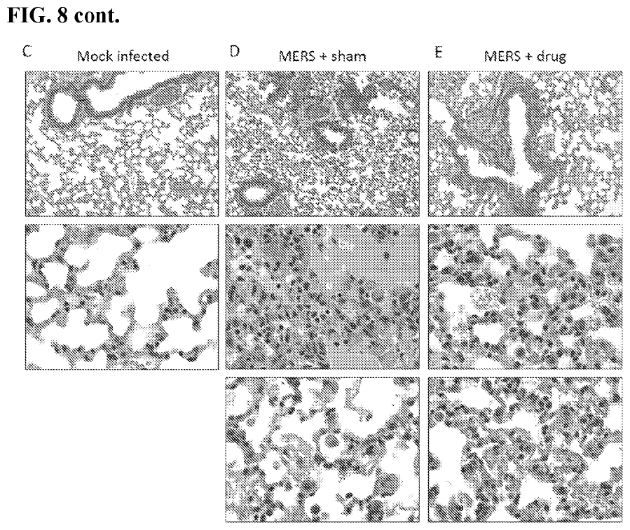

PERK inhibition improves pulmonary function and diminishes MERS-CoV pathogenesis and ALI. Because treatment of primary human lung cells with PERK AMG PERK 44 inhibitor resulted in a significant decrease in MERS-CoV replication in both cell types, we sought to determine if in vivo inhibition would result in a change in viral replication and/or pathogenesis. To address this, a transgenic mouse model was used where the murine ortholog of the human MERS-CoV receptor, dipeptidyl peptidase 4 (DPP4), was humanized at residues 288 and 330 (hDPP4) facilitating high titer virus replication localized to the respiratory tract and lung pathology similar to that observed in humans. hDPP4 mice were treated with AMG PERK 44 or vehicle at 24 hours prior to and post MERS-CoV infection. By three days post MERS-CoV infection, vehicle treated mice lost significantly (p value <0.05) more weight than AMG PERK 44 treated mice. Importantly, AMG PERK 44 treated mice also displayed an improved time to recovery over the remainder of the study (FIG. 7 panel A). Interestingly, virus titer in the lung on day 3 post infection was similar regardless of treatment (FIG. 7 panel B). “Pulmonary discoloration score,” a gross pathological phenotype that increases coincident with MERS-CoV pathogenesis, was significantly diminished in AMG PERK 44 treated mice as compared to those receiving vehicle treatment (FIG. 7 panel C). To determine if PERK inhibition through AMG PERK 44 treatment would affect pulmonary function, whole body plethysmography (WBP) was performed daily. Concordant with weight loss and pulmonary hemorrhage, AMG PERK 44 treatment improved pulmonary function as compared to those receiving vehicle. For three WPB metrics typically induced following MERS-CoV infection, Pause, Penh and EF50, AMG PERK 44 treated mice had significantly improved values (p value <0.05) than vehicle treated animals at multiple timepoints (FIG. 7 panels D-F). Pause, PenH and EF50 are all measures of airway constriction or obstruction and indicate that AMG PERK 44 treatment is relieving or preventing lung pathology that negatively affects lung function. To quantitatively assess the impact of AMG PERK 44 treatment on MERS-CoV infected mice, histological lung sections were assessed using the American Thoracic Society (ATS) scoring system designed to more closely relate data from small animal models of acute lung injury (ALI) to infected patient outcomes. Hematoxylin and eosin stained fields of lung tissue were blindly scored for the presence of neutrophils in the alveolar and interstitial space, hyaline membranes, proteinaceous debris filling the airway spaces, and thickening of the alveolar septa. ATS lung injury scores were significantly reduced in male but not female MERS-CoV infected AMG PERK 44 treated mice (FIG. 8 panels A-E).

Materials and methods. Primary human lung cells were isolated from distal lung tissue and processed. Human lung microvascular endothelial cells (MVE) were cultured in Vasculife VEGF-MVE Endothelial Media (Lifeline Cell Tech). Human lung fibroblasts (FB) were cultured in DMEM-H Basal Medium (Corning), ix penicillin/streptomycin (Sigma), 10% fetal bovine serum (Gemini Bio). Media fetal bovine serum concentrations were reduced to 4% prior to infection. Vero 81 cells were cultured in Dulbecco's modified essential media (DMEM Gibco) with 10% fetal clone II (Hyclone) and 1× antibiotic/antimycotic (Gibco).

Viruses and viral titration Wild-type MERS-CoV (EMC 2012 strain), MERS-CoV expressing nano luciferase (MERS nanoluc) and MERS-CoV expressing the red fluorescent protein (MERS-RFP) were rescued from infectious clones. Vero C81 cells were used to generate viral stocks and to quantitate viral titers following infection.

Donor matched MVE and FB from three different human tissue donors were infected and samples harvested for systems biology analysis. For transcriptomic analysis, five replicate wells of MVE or FB were either mock-infected or infected with wild type MERS-CoV at multiplicity of infection (MOI) 5. At 0, 12, 24, 36, and 48 hours post infection, 100 of media/supernatant was collected for viral titration assays, media removed and then 1 mL of Trizol (Invitrogen/ThermoFisher) added per well. Total RNA samples were sent to Arraystar for downstream analysis. In parallel, an additional five replicate wells were subjected to the MPLEx protocol (chloroform/methanol, 2:1 mix) precipitation, which results in simultaneous extraction of proteins, metabolites, and lipids with concomitant inactivation of virus. All proteomic and lipidomic samples were desiccated and frozen prior to downstream analysis.

Scanned images were analyzed using Agilent Feature Extraction Software (v11.0.1.1). The limma package for R (available on Bioconductor) was used to perform background correction, quantile normalization (normalizeBetweenArrays), and summarization (avereps) to derive a single normalized intensity value per probe. Outlier samples were detected using PCA and by visual inspection of heatmaps, and all data was re-processed after removing outlier samples. All data processing for each of the biological replicates was performed independently of the other.

Samples for proteomics analysis were prepared for and analyzed using liquid chromatography-mass spectrometry and the Accurate Mass and Time Tag approach. The RMD-PAV algorithm (Matzke et al. 2011 Bioinformatics, 27:2866-2872) was used to identify any outlier biological samples and was confirmed via Pearson correlation. Peptides with inadequate data for either qualitative or quantitative statistical tests were also removed from the dataset. The SPANS algorithm (Webb-Robertson et al. 2010 J Proteome Res 9:5748-5756) was used to identify the best normalization method for each dataset. Peptides were evaluated with Analysis of Variance (ANOVA) with a Dunnett test correction and a Bonferroni-corrected g-test to compare each virus to the associated mock within each time point. To perform signature-based protein quantification, BP-Quant (Webb-Robertson et al. 2014 Mol Cell Proteomics 13:3639-3646), each peptide was categorized as a vector of length equal to the number of viruses being evaluated. If all comparisons for all time points are 0 for a specific virus it is considered as non-changing and given a value of 0. If there are more time points with an increase in virus to mock than decreasing it is categorized as a +1 and the contrary −1 is given for the decrease in virus to mock. BP-Quant was run with a default parameter of 0.9. All proteins were then analyzed using the same methodology as for the peptides; ANOVA with a Dunnett test correction and a Bonferroni-corrected g-test to compare each virus to the associated mock within each time point.

Samples for lipidomics analysis were prepared for and analyzed using liquid chromatography-tandem mass spectrometry. Lipids were identified using the tool LIQUID (Kyle et al. 2017 Bioinformatics 33:1744-1746), and their quantitative data were extracted using MZmine 2.0. The RMD-PAV algorithm was also used to identify any outlier biological samples and was confirmed via Pearson correlation. Lipids with inadequate data for either qualitative or quantitative statistical tests were also removed from the dataset. Median centering was used for normalization. Lipids were evaluated with a standard two sample t-test to compare each infected condition to the associated mock within each time point.

All datasets used for these analyses can be found at data.pnnl.gov under the following numbers. MERS-CoV infected fibroblasts-13095 (FB donor 1), 13096 (FB donor 2), 13097 (FB donor 3), MERS-CoV infected microvascular endothelial cells 13102 (MVE donor 1), 13103 (MVE donor 2), and 13104 (MVE donor 3), MERS-CoV infected human airway epithelial cell cultures (HAE) 1661938 (HAE donor 1), 1661939 (HAE donor 2), 1661940 (HAE donor 3).

Functional enrichment identified significantly changed features (transcripts, proteins, lipids) by using an adjusted p-value of 0.05 as a threshold. For each experimental condition, perturbed features were divided into one list each of up- and down-regulated items. Each list was tested against the Gene Ontology (GO) database of gene sets using the EASE-adjusted one-sided Fisher exact test, such that each condition is tested for up- and down-regulation of each function/pathway in the ontology database. Results across multiple conditions were visualized in heatmaps using the log 10 p-values of the significance tests, colored to indicate the direction of change. For lipids, feature sets were generated by categorizing the lipids observed in our experiments according to broad lipid classifications; these classifications were then used as “gene sets” and the enrichment analysis was then performed in the same manner as for transcripts and proteins.

To assess numbers of viable cells following MERS-CoV infection of FB and MVE, CellTiter-Glo Luminescent Cell Viability Assay (Promega) was used at 24 and 48 hours post infection/treatment according to manufacturer's instructions and relative light units were determined by Spectromax plate reader (Molecular Devices), averaged and plotted. Percent infection was determined using MVE and FB infected with MERS-RFP (MOI 5) or mock-infected and counting nuclei (Horcht stain diluted 1:5,000 in media) in replicate wells at 0, 12, 24, 36, and 48 hours post infection. Cell numbers were determined using the Fiji cell counter function and total number of cells versus number of red fluorescent cells were determined for at least three wells per condition per cell type. Statistical significance was determined by Mann-Whitney U test.

Infected FB and MVE (MOI 5 wild type MERS-CoV) were assayed using either the Caspase-Glo 3/7 Kit (Promega) to measure caspase activation or using the ApoTox-Glo Triplex Assay (Promega) to assess cell viability, cytotoxicity and caspase 3/7 activation in a single set of samples at 24 and 48 hours post infection according to manufacturer's instructions. Control wells were treated with UV inactivated virus stocks, staurosporin (Sigma, 8 μM for MVE or 10 μM for FB) or ionomycin (ThermoFisher, 40 μM for MVE and 60 μM for FB). Relative light units or emission excitation wavelengths were determined by Spectromax plate reader (Molecular Devices). Statistical significance was determined by Mann-Whitney U test.

For inhibitor studies, MERS-CoV infected MVE and FB were tested at 24 and 48 hours post infection following treatment with a dilution series of trans-ISRIB (0.5 μM to 0.00488 μM) or PERK inhibitor (Tocris, AMG PERK 44, 100 μM to 2 μM) and was evaluated for cytotoxicity with drug treatment alone (cell viability assay, no virus), activation of the death caspases 3/7 (wild type MERS-CoV, MOI 5) and viral replication (detection of luciferase as a surrogate for replication, MERS nanoluc virus, MOI 5). All assay results were read by SpectraMax (Molecular Devices). Results are graphed as percent inhibition and toxicity (effective and cytotoxic concentration 50) or fold change above mock (caspase activation) as determined by GraphPad Prism.

For mouse studies, hDPP4 mice were used. For a single group of mice, dosing occurred once a day 24 hours prior to infection and a second single dose was administered 24 hours post infection via the intraperitoneal route (12 mg/kg of PERK AMG PERK 44 (Tocris)) suspended in water or with a sham control). Immediately prior to infection, mice were anesthetized with 50 μL of ketamine/xylazine mixture via intraperitoneal injection. 16-20 week old mice were intranasally infected with 10⁴ PFU of mouse adapted MERS-CoV maM35c4 diluted in OPTIMEM, a total volume of 50 μL was given. Mice were weighed daily following infection and had their respiratory function measured one day prior to infection and at days two through six post infection using Buxco Whole Body Plethysmography (Data Sciences International). Briefly, mice were allowed to acclimate for 30 minutes in the plethysmography chamber located within the biological safety cabinet after which measurements were taken for 5 minutes. Readings were collected every 2 seconds for a total of 150 measurements per mouse per day. At days three and seven post infection mice were euthanized via isoflurane overdose after which lung tissue was harvested to determine viral replication titers. Gross pulmonary hemorrhage was observed and scored from zero (none) to four (severe and total) at the time of dissection.

All statistical data analyses were performed in using Graphpad Prism 8 software. Statistical significance for each endpoint was determined with specific statistical tests. For each test, a p-value <0.05 was considered significant. Specific tests are noted in each figure legend.

All datasets used for these analyses can be found at data.pnnl.gov under the following numbers. MERS-CoV infected fibroblasts-13095 (FB donor 1), 13096 (FB donor 2), 13097 (FB donor 3), MERS-CoV infected microvascular endothelial cells 13102 (MVE donor 1), 13103 (MVE donor 2), and 13104 (MVE donor 3), MERS-CoV infected human airway epithelial cell cultures (HAE) 1661938 (HAE donor 1), 1661939 (HAE donor 2), 1661940 (HAE donor 3).

All references cited herein are incorporated herein by reference in their entireties and for all purposes to the same extent as if each individual publication or patent or patent application was specifically and individually indicated to be incorporated by reference in its entirety for all purposes.

The foregoing examples are illustrative of the present invention, and are not to be construed as limiting thereof. Although the invention has been described in detail with reference to preferred embodiments, variations and modifications exist within the scope and spirit of the invention as described and defined in the following claims.

1. A method for treating a coronavirus infection in a subject, comprising administering to the subject an effective amount of an inhibitor of an unfolded protein response (UPR), an inhibitor of an integrated stress response (ISR), and/or an inhibitor of protein kinase RNA-like endoplasmic reticulum kinase (PERK).

2. A method for treating a disease or disorder caused by or associated with a coronavirus infection in a subject, comprising administering to the subject a therapeutically effective amount of an inhibitor of an unfolded protein response (UPR), an inhibitor of an integrated stress response (ISR), and/or an inhibitor of protein kinase RNA-like endoplasmic reticulum kinase (PERK).

3. The method of claim 1, wherein the coronavirus is Middle East respiratory syndrome coronavirus (MERS-CoV), severe acute respiratory syndrome coronavirus (SARS-CoV) or severe acute respiratory syndrome coronavirus 2 (SARS-CoV-2).

4. The method of claim 1, wherein the inhibitor is selected from the group consisting of GSK2606414 (PERK inhibitor), GSK2656157 (PERK inhibitor), ISRIB (trans-isomer) (PERK inhibitor), Salubrinal inhibitor of eIF-2α dephosphorylation and inhibitor of stress-mediated apoptosis), Sal003 (inhibitor of eIL-2α phosphatase), Azoramide (inhibitor of unfolded protein response (UPR)), AMG PERK 44 (PERK inhibitor), PERK-IN-2 (PERK inhibitor), PERK-IN-3 (PERK inhibitor), 4μ8C-CAS 14003-96-4-Calbiochem (IRE1 inhibitor III), CAS 608512-97-6-Calbiochem (PKR inhibitor), STF-083010-Calbiochem (IRE1 inhibitor), KIRA6-Calbiochem (IRE1 inhibitor), LDN-0070977 (PERK inhibitor III), NMS-E194, AMG-44, trazodone HCl, any derivative of any of said inhibitors, and any combination of said inhibitors and derivatives.

5. The method of claim 1, wherein said inhibitor inhibits viral activity by at least 50%.

6. The method of claim 1, wherein said inhibitor is administered in a range of about 0.1 mg/kg to about 500 mg/kg per day.

7. The method of claim 1, further comprising administering an antiviral drug.

8. The method of claim 7, wherein the antiviral drug is interferon, ribavirin, adefovir, tenofovir, acyclovir, brivudin, cidofovir, fomivirsen, foscarnet, ganciclovir, penciclovir, amantadine, rimantadine and/or zanamivir, singly or in any combination.

9. The method of claim 2, wherein the coronavirus is Middle East respiratory syndrome coronavirus (MERS-CoV), severe acute respiratory syndrome coronavirus (SARS-CoV) or severe acute respiratory syndrome coronavirus 2 (SARS-CoV-2).

10. The method of claim 2, wherein the inhibitor is selected from the group consisting of GSK2606414 (PERK inhibitor), GSK2656157 (PERK inhibitor), ISRIB (trans-isomer) (PERK inhibitor), Salubrinal inhibitor of eIF-2α dephosphorylation and inhibitor of stress-mediated apoptosis), Sal003 (inhibitor of eIL-2α phosphatase), Azoramide (inhibitor of unfolded protein response (UPR)), AMG PERK 44 (PERK inhibitor), PERK-IN-2 (PERK inhibitor), PERK-IN-3 (PERK inhibitor), 4μ8C-CAS 14003-96-4-Calbiochem (IRE1 inhibitor III), CAS 608512-97-6-Calbiochem (PKR inhibitor), STF-083010-Calbiochem (IRE1 inhibitor), KIRA6-Calbiochem (IRE1 inhibitor), LDN-0070977 (PERK inhibitor III), NMS-E194, AMG-44, trazodone HCl, any derivative of any of said inhibitors, and any combination of said inhibitors and derivatives.

11. The method of claim 2, wherein said inhibitor inhibits viral activity by at least 50%.

12. The method of claim 2, wherein said inhibitor is administered in a range of about 0.1 mg/kg to about 500 mg/kg per day.

13. The method of claim 2, further comprising administering an antiviral drug.

14. The method of claim 13, wherein the antiviral drug is interferon, ribavirin, adefovir, tenofovir, acyclovir, brivudin, cidofovir, fomivirsen, foscarnet, ganciclovir, penciclovir, amantadine, rimantadine and/or zanamivir, singly or in any combination.
